(19) United States
(12) Patent Application Publication
(10) Pub. No.: US 2009/0318937 A1
(43) Pub. Date: Dec. 24, 2009

(54) CLIP COUPLING METHOD AND MULTIPLE CLIP PACKAGE

(75) Inventors: Yoshiaki MATSUOKA, Saitama (JP); Takayuki IIDA, Saitama (JP); Shengfu Cui, Saitama (JP); Yoshiyuki KUNUKI, Saitama (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 12/489,898

(22) Filed: Jun. 23, 2009

(30) Foreign Application Priority Data
Jun. 24, 2008 (JP) 2008-164687
Jun. 24, 2008 (JP) 2008-164688
Jul. 7, 2008 (JP) 2008-177162
Jul. 7, 2008 (JP) 2008-177163
Sep. 11, 2008 (JP) 2008-233655
Oct. 8, 2008 (JP) 2008-261526

(51) Int. Cl. A61B 17/10; A61B 17/08

(52) U.S. Cl. 606/143; 606/151

(57) ABSTRACT

A multiple hemostatic clip application apparatus includes a multiple clip assembly and a flexible sheath coupled with the multiple clip assembly, and operates for tissue clamping in combination with an endoscope. For the clip coupling, a pull rod structure is pulled to introduce the multiple clip assembly into a fin bending channel in a housing. Fins of the multiple clip assembly are depressed and stowed by the inside of the fin bending channel. The housing is removed from a coupling device. An operating wire is fastened to a fastening clip device. The flexible sheath is inserted in an access hole to register the inside of the flexible sheath with the fin bending channel. When the operating wire is pulled relative to the flexible sheath, the multiple clip assembly is introduced into the flexible sheath.

### BACKGROUND OF THE INVENTION

1. Field of the Invention

The present invention relates to a clip coupling method and a multiple clip package. More particularly, the present invention relates to a clip coupling method and a multiple clip package, in which a multiple clip assembly including plural clips and tubular shells is used, and offsetting of tubular shells from clips due to fins is prevented in the course of clip coupling of the multiple clip assembly into a flexible sheath.

2. Description Related to the Prior Art

Tissue clamping is known as medical treatment of a lesion in a gastrointestinal tract by use of an endoscope. In the clamping, a clip of a small size is used to clamp the lesion for the purpose of hemostasis, suture and the like. A hemostatic clip application apparatus for the tissue clamping is entered in the body through a forceps channel of an endoscope, and clamps the tissue with the clip at a distal end thereof. U.S. Pat. No. 6,814,742 (corresponding to JP-A 2007-222649) discloses the hemostatic clip application apparatus including a flexible sheath, an operating wire and a handle device. The flexible sheath is loaded with the clip. The operating wire is fastened to a proximal end of the clip in the flexible sheath. The handle device is adapted to operating the flexible sheath and the operating wire.

In U.S. Pat. No. 6,814,742, a push sleeve is used to drive the distal end of the clip. Claws of the clip are set by the push sleeve in a closed state. The push sleeve is loaded in the flexible sheath together with the clip in a state secured to the proximal end of the clip, and moves forwards relative to the clip for the tissue clamping, to close the distal end of the clip by pushing. Fins or skirts are formed with the outside of the push sleeve, are closed when contained in the flexible sheath, and are open radially with their resiliency when moved out of the flexible sheath with the clip, so as to prevent the clip from moving back by engagement of the distal end of the flexible sheath.

U.S. Pat. No. 6,814,742 also discloses a housing for preserving an unused clip with which the push sleeve is mounted. In the housing, the clip is contained in a state of opening the fins for the purpose of preventing a drop of resiliency of the fins. An inclined surface is formed with an exit opening of the housing for closing the fins. For loading of the clip in the flexible sheath from the housing, a shaft head for hooking at the distal end of the operating wire is inserted in the housing through the exit opening, and is engaged with the clip having the push sleeve. The operating wire is pulled to move back the clip and introduce the clip into the flexible sheath through the exit opening of the housing. In the introduction of the clip from the housing into the flexible sheath, the fins are contacted and closed by the inclined surface.

Only one clip can be loaded in the hemostatic clip application apparatus of known types disclosed in U.S. Pat. No. 6,814,742 and others. It is necessary to unload the hemostatic clip application apparatus from an endoscope after one time of the tissue clamping and then to insert the hemostatic clip application apparatus in the endoscope after coupling of the clip being unused.

In view of such a problem, JP-A 2006-187391 discloses a multiple hemostatic clip application apparatus in which the tissue clamping of a consecutive manner is possible. Connection holes are formed in the proximal end of a first one of the clips. A second one of the clips has the claws at the distal end. The claws are engaged with the connection holes to fasten the clips to one another directly with differences of the direction at 90 degrees in an alternate manner.

Also, JP-A 2008-049198 discloses the multiple hemostatic clip application apparatus including a train of the clips and a plurality of the push sleeves. The push sleeves are contained in the flexible sheath with the clips in association. The push sleeves have the fins in a manner similar to that of U.S. Pat. No. 6,814,742.

In the multiple hemostatic clip application apparatus of JP-A 2006-187391, only engagement between the clips maintains the fastened state between those. Their fastened portion is uncovered. There is a problem in that the fastened state is unstable, and the fastened portion is likely to disengage in the course of passage in a tortuous portion of a tube of an endoscope during the insertion. Also, distortion or deformation is likely to occur with the clips before use due to overstress applied to the fastened portion.

A new type of the multiple hemostatic clip application apparatus has been developed to solve the problem of JP-A 2006-187391. The multiple hemostatic clip application apparatus includes a train of the clips and tubular shells or retaining rings. The clips are fastened to one another by engagement of the claws of the distal end with the proximal end in a closed state. The tubular shells are disposed around the clips, cover the claws of the clips on a proximal side, and maintain their fastened state. The tubular shells also have the fins in the same manner as the push sleeve in U.S. Pat. No. 6,814,742. When the tubular shells advance relative to the clips, the distal end of one of the tubular shells pushes the distal end of one of the clips to close the claws.

The clips and the tubular shells must be set in the flexible sheath in a fastened state. It is difficult for an operator to fasten the clips before loading in the flexible sheath due to restraint of time of operation. In view of this, there is a suggestion of a multiple clip assembly produced by previously assembling the clips and the tubular shells. The multiple clip assembly is contained in the housing as an article for supply. Also, the fins of the tubular shells formed from plastic material should be prevented from deforming plastically. Thus, the multiple clip assembly is contained in the housing in which the fins remain open.

The fins of the tubular shells must be closed for operation of loading the multiple clip assembly in the flexible sheath. It is conceivable to utilize the method of U.S. Pat. No. 6,814,742. The fins of the tubular shells may be closed by means of the inclined surface in the housing for loading the multiple clip assembly from the housing into the flexible sheath.

In the clip coupling of U.S. Pat. No. 6,814,742, relative offsetting between the tubular shells and the clips is likely to occur as resistance of friction is caused by closing of the fins on the inclined surface of the housing. The suggested structure is not usable for loading of the multiple clip assembly. This is because higher precision in the position of the clips relative to the tubular shells is required than the hemostatic clip application apparatus of a single clip. Specifically, the clips and the tubular shells are advanced through the distal end of the flexible sheath one after another serially by the pull of the flexible sheath at a predetermined amount in the hemostatic clip application apparatus. Furthermore, JP-A 2008-049198 does not suggest a specific method of mounting the push sleeve on the flexible sheath in which the push sleeve has the fins.

### SUMMARY OF THE INVENTION

In view of the foregoing problems, an object of the present invention is to provide a clip coupling method and a multiple clip package, in which a multiple clip assembly including plural clips and tubular shells is used, and offsetting of tubular shells from clips due to fins is prevented in the course of clip coupling of the multiple clip assembly into a flexible sheath.

In order to achieve the above and other objects and advantages of this invention, a clip coupling method of loading a multiple clip assembly in a flexible sheath of a tubular shape is provided, wherein the multiple clip assembly includes a plurality of clips arranged in one train and fastened to one another, and a plurality of tubular shells loaded in the flexible sheath together with respectively the clips, and the tubular shells have a fin portion for deploying with resiliency upon advance through a sheath end of the flexible sheath with one of the clips, and for engaging with the sheath end, one of the tubular shells shifts and closes one of the clips when the clips move back toward an inside of the flexible sheath. The clip coupling method includes a step of introducing the multiple clip assembly from a housing into a fin bending channel by advancing a distal end thereof, the housing containing the multiple clip assembly in a state of deploying the fin portion, the fin bending channel having an inner diameter substantially equal to an inner diameter of an end opening of the sheath end of the flexible sheath in connection of a coupling device with the housing, the fin bending channel depressing and stowing the fin portion. The housing is removed from the coupling device, to fasten a distal end of an operating wire inserted movably through the flexible sheath to a proximal end of the multiple clip assembly. The end opening of the flexible sheath is positioned in registration with the fin bending channel. The operating wire is pulled relative to the flexible sheath, to introduce the multiple clip assembly into the housing by advancing the proximal end thereof in a state of depressing the fin portion.

The multiple clip assembly is introduced from the housing into the coupling device by pull of a first one of the clips.

The multiple clip assembly is introduced from the housing into the coupling device by push of a rear one of the clips.

The tubular shells are disposed around respectively the clips, partially cover a rear one of the clips for maintaining a fastened state thereof, and are prevented from moving backwards by the rear clip.

The fin portion pushes and retains one of the clips in the tubular shells when depressed and stowed.

In one aspect of the invention, a multiple clip package including a multiple clip assembly is provided, wherein the multiple clip assembly has a plurality of clips arranged in one train and fastened to one another, and a plurality of tubular shells loaded in a flexible sheath of a tubular shape together with respectively the clips, and the tubular shells have a fin portion for deploying with resiliency upon advance through a sheath end of the flexible sheath with one of the clips, and for engaging with the sheath end, one of the tubular shells shifts and closes one of the clips when the clips move back toward an inside of the flexible sheath, the fin portion is deployed while the multiple clip assembly is contained, and is depressed and stowed by loading of the multiple clip assembly in the flexible sheath. The multiple clip package includes a housing, having a barrel cavity for containing the multiple clip assembly, the barrel cavity having an inner diameter substantially equal to an inner diameter of an end opening of the sheath end of the flexible sheath, and including a fin receiving opening or recess and an exit opening, the fin receiving opening or recess containing the fin portion of the multiple clip assembly in a deployed state, the exit opening causing the multiple clip assembly to move to an outside of the barrel cavity by advancing a distal end of the multiple clip assembly. A coupling device has a stage portion and a fin bending channel, the stage portion receiving the housing mounted thereon, the fin bending channel having an inner diameter substantially equal to an inner diameter of the barrel cavity, wherein the fin bending channel is positioned in registration with the exit opening of the barrel cavity upon mounting the housing on the stage portion, and depresses and stows the fin portion upon introduction of the multiple clip assembly from the barrel cavity.

The coupling device includes a clip moving structure for introducing the multiple clip assembly from the barrel cavity into the fin bending channel by advancing a distal end thereof.

The clip moving structure includes a pull tab portion pullable relative to the coupling device. A shank has the pull tab portion positioned at a first end thereof, and inserted in the fin bending channel through a pull opening formed in the coupling device. An end connector is disposed at a second end of the shank, for engaging with a first one of the clips in the multiple clip assembly, and for drawing the first clip when the pull tab portion is pulled, to introduce the multiple clip assembly from the barrel cavity into the fin bending channel.

A first one of the clips in the multiple clip assembly is engaged with the end connector by keeping claws closed with pressure of an inner surface of the barrel cavity, the claws being open when in a free state without receiving external force. The fin bending channel has a release groove for shifting the first clip to open with resiliency of the first clip when the multiple clip assembly is introduced and set in a predetermined position in the fin bending channel, to disengage the first clip from the end connector.

An opening direction of the first clip is different from a deploying direction of the fin portion of the tubular shell associated with the first clip and with a difference of substantially a ¼ rotation with respect to an axial direction of the tubular shell. The release groove extends in the opening direction of the first clip.

The multiple clip assembly includes a fastening clip for engaging with a rear one of the clips. There is a fastening mechanism for supporting the fastening clip and for fastening to an operating wire inserted in the flexible sheath.

The coupling device includes a wire channel and a connection opening for receiving insertion of the operating wire and the shaft head transversely to the axial direction of introducing the multiple clip assembly into the fin bending channel. The fastening mechanism is positioned at the connection opening when the multiple clip assembly is introduced in the fin bending channel.

Furthermore, a guide mechanism pushes the shaft head inserted in the connection opening, for fastening to the fastening mechanism.

The guide mechanism is a slider for sliding transversely to the axial direction of the multiple clip assembly in the connection opening positioned between the stage portion and the fin bending channel, and for pushing the shaft head in the connection opening upon sliding, to fasten the shaft head with the fastening mechanism.

The slider includes a receiving bore having a diameter substantially equal to a diameter of the fin bending channel.

The receiving bore is disposed between the fin bending channel and the stage portion when the slider is slid for fastening the shaft head to the fastening mechanism.

The housing is cylindrical and has an outer diameter substantially equal to an outer diameter of the flexible sheath, and the stage portion is loaded with the flexible sheath after removal of the housing.

An opening of a sheath end of the flexible sheath on the stage portion is positioned in registration with the fin bending channel, and the multiple clip assembly pulled with the operating wire is loaded in the flexible sheath by advancing a proximal end thereof in a state of stowing the fin portion.

The coupling device includes a stage groove formed for causing the housing or the flexible sheath on the stage portion to appear externally at least partially. A recess is formed in a peripheral portion of the stage groove, for protruding a portion of the housing or the flexible sheath appearing at least partially.

In one preferred embodiment, the tubular shells are disposed around respectively the clips, partially cover a rear one of the clips for maintaining a fastened state thereof, and are prevented from moving backwards by the rear clip.

The fin portion pushes and retains one of the clips in the tubular shells when depressed and stowed.

In another preferred embodiment, furthermore, an engaging portion is disposed with a first one of the housing and the stage portion. A receiving portion is disposed with a second one of the housing and the stage portion, for preventing the housing from rotating with respect to the coupling device by engagement with the engaging portion.

A first one of the engaging portion and the receiving portion is a key projection disposed on the housing to project, and a second one of the engaging portion and the receiving portion is a key way groove formed in the stage portion to extend in an axial direction of the housing.

Furthermore, a retaining mechanism positions the key projection engaged with the key way groove in a predetermined position in the axial direction, for retaining the housing to prevent separation in the axial direction.

The retaining mechanism includes a retaining projection formed to project from a first one of the key projection and the key way groove. A retaining hole or recess is formed with a second one of the key projection and the key way groove.

In one preferred embodiment, the shank has a shape of a section engageable with the pull opening rotationally with respect to an axial direction of insertion thereof.

The shank has resiliency for absorbing twisting of the pull tab portion with respect to the axial direction between the pull tab portion and the pull opening.

The shank includes at least two resilient elongated plates, disposed to extend in the axial direction, and arranged at a predetermined interval in a direction transverse to the axial direction, and a shape of a section of the shank defined by a contour of the elongated plates is similar to a shape of the pull opening.

The elongated plates have such a form that the shank is engaged with the pull opening rotationally when the elongated plates are deformed toward one another.

In still another preferred embodiment, furthermore, a retainer is disposed in the pull opening. A receiving portion is disposed with the shank, for retaining the clip moving structure by engagement with the retainer when the clip moving structure is in a predetermined position with respect to the coupling device.

The coupling device includes a releasing portion for disengaging the first clip from the end connector when a fastening mechanism at a proximal end of the multiple clip assembly reaches a position for enabling fastening to an operating wire in the flexible sheath. The receiving portion is positioned for engaging with the retainer in operation of the releasing portion.

The receiving portion is positioned for engaging with the retainer when the clip moving structure is pulled to an end position in the coupling device.

The receiving portion is positioned for engaging with the retainer when the clip moving structure is in an initial position before pull relative to the coupling device.

The shank includes at least two resilient elongated plates, disposed to extend in an axial direction of insertion thereof, and arranged at a predetermined interval in a direction transverse to the axial direction. The receiving portion includes a retaining hole formed in at least one of the elongated plates, and the retainer includes a projection for engaging with the retaining hole, and for disengagement from the retaining hole when the elongated plates resiliently deform in a direction toward one another.

Accordingly, offsetting of tubular shells from clips due to fins can be prevented in the course of clip coupling of the multiple clip assembly into a flexible sheath.

### BRIEF DESCRIPTION OF THE DRAWINGS

The above objects and advantages of the present invention will become more apparent from the following detailed description when read in connection with the accompanying drawings, in which:

FIG. 1 is a perspective view illustrating a multiple hemostatic clip application apparatus;

FIG. 2A is a section illustrating a flexible sheath in the multiple hemostatic clip application apparatus at its sheath end;

FIG. 2B is a section illustrating the same as FIG. 2A but viewed perpendicularly to a direction of FIG. 2A;

FIG. 3 is a perspective view illustrating a clip device and a tubular shell;

FIG. 4 is a section illustrating the tubular shell;

FIG. 5 is a bottom plan illustrating the tubular shell;

FIG. 6 is a perspective view illustrating a final one of the clip devices, a fastening clip device and an operating wire;

FIG. 7A is a section illustrating a first step in a sequence of tissue clamping with the multiple hemostatic clip application apparatus;

FIG. 7B is a section illustrating a step of opening the clip device in the clamping sequence;

FIG. 7C is a section illustrating tissue clamping in the clamping sequence;

FIG. 7D is a section illustrating a step of separating the clip device from second and other clip devices;

FIG. 8 is a perspective view illustrating a first preferred multiple clip package;

FIG. 9 is a section illustrating the multiple clip package;

FIG. 10 is an exploded perspective view illustrating the multiple clip package;

FIG. 11 is a perspective view illustrating a multiple clip assembly, a housing and a pull rod structure;

FIG. 12 is a bottom perspective view illustrating the multiple clip package;

FIG. 13 is an exploded perspective view illustrating a coupling device;

FIG. 14 is a perspective view illustrating an area on the coupling device with a key way groove;

FIG. 15 is a section illustrating a release groove of the coupling device;

FIGS. 16A and 16B are sections illustrating disengagement of the multiple clip assembly from the pull rod structure with the release groove;

FIG. 17 is a perspective view illustrating a slider;

FIGS. 18A and 18B are sections illustrating insertion of a shaft head into an engaging portion;

FIGS. 19A and 19B are sections illustrating fastening of the shaft head to the engaging portion with a second inclined surface;

FIGS. 20A and 20B are sections illustrating introduction of the multiple clip assembly from the housing into the coupling device and removal of the housing from the coupling device;

FIGS. 21A and 21B are sections illustrating insertion of the flexible sheath into the coupling device to fasten the multiple clip assembly to the operating wire;

FIGS. 22A and 22B are sections illustrating insertion of the flexible sheath into the coupling device and introduction of the multiple clip assembly from the coupling device into the flexible sheath;

FIG. 23 is a section illustrating fins in the introduction of the multiple clip assembly into a fin bending channel;

FIG. 24 is a section illustrating the fins during introduction of the multiple clip assembly from the fin bending channel into the flexible sheath;

FIG. 25 is a perspective view illustrating a second preferred embodiment of the multiple clip package;

FIGS. 26A and 26B are sections illustrating the multiple clip assembly fastened to an operating wire, and pushed into the coupling device;

FIGS. 27A and 27B are sections illustrating separation of the housing and insertion of the flexible sheath into the coupling device;

FIG. 28 is a perspective view illustrating a third preferred embodiment of the coupling device for the multiple clip package;

FIG. 29 is a section illustrating an unused state of the multiple clip package;

FIG. 30 is a section illustrating a slide channel of the multiple clip package;

FIG. 31 is a perspective view illustrating the slider;

FIG. 32 is a section illustrating introduction of the multiple clip assembly into the flexible sheath;

FIG. 33 is a section illustrating an inner position of the slider in a slide channel;

FIG. 34 is an exploded perspective view illustrating a fourth preferred embodiment of the multiple clip package;

FIG. 35 is a perspective view illustrating the pull rod structure;

FIG. 36 is a perspective view illustrating a pull opening of the coupling device;

FIG. 37 is a section illustrating engagement of a shank with the pull opening as viewed in the axial direction;

FIG. 38A is a section illustrating the engagement of the shank with the pull opening;

FIG. 38B is a section illustrating disengagement of the shank from the pull opening;

FIG. 39 is a section illustrating disengagement of the clip device from an engaging portion with the release groove;

FIG. 40 is a perspective view illustrating a fifth preferred embodiment in which a retaining projection is disposed on a key projection;

FIG. 41 is a perspective view illustrating a key way groove and a retaining hole formed in combination.

### DETAILED DESCRIPTION OF THE PREFERRED EMBODIMENT(S) OF THE PRESENT INVENTION

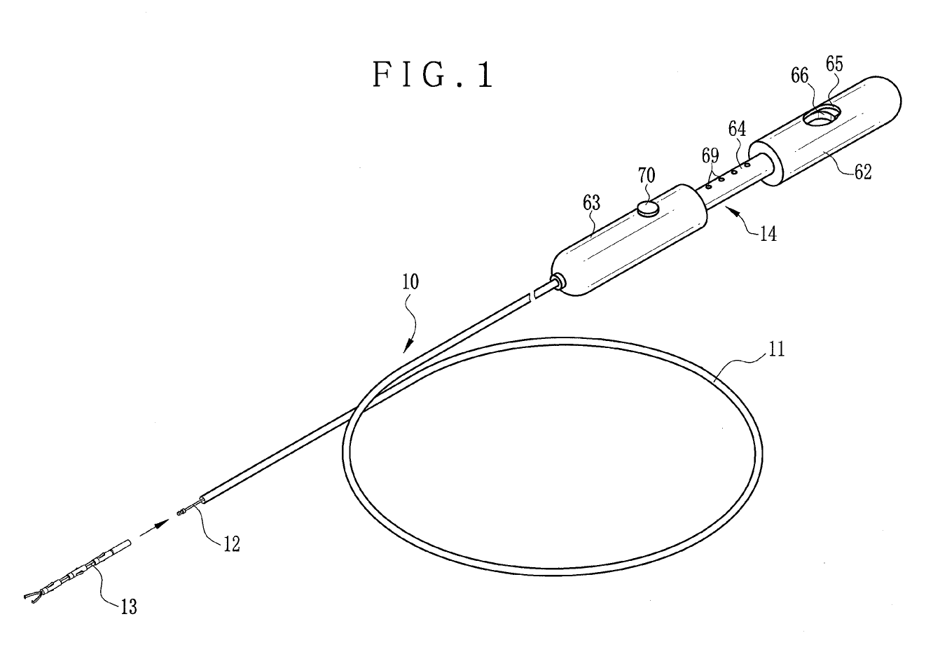

In FIG. 1, a multiple hemostatic clip application apparatus 10 of the invention is illustrated. The multiple hemostatic clip application apparatus 10 includes a cylindrical flexible sheath 11, an operating wire 12, a multiple clip assembly 13 or clip train, and a handle device 14.

The flexible sheath 11 has a great length. The operating wire 12 is inserted through the flexible sheath 11 movably back and forth. The multiple clip assembly 13 includes a train of plural clips fastened to one another. The multiple clip assembly 13 is contained in a portion of a sheath end of the flexible sheath 11. A proximal end of the multiple clip assembly 13 is fastened to a distal end of the operating wire 12. The handle device 14 is connected with proximal ends of the flexible sheath 11 and the operating wire 12, and is pulled manually. When the flexible sheath 11 is pulled, each of the clips in the multiple clip assembly 13 is pushed forwards through the sheath end. The clip is open when pushed out of the flexible sheath 11, and is closed when the operating wire 12 is pulled.

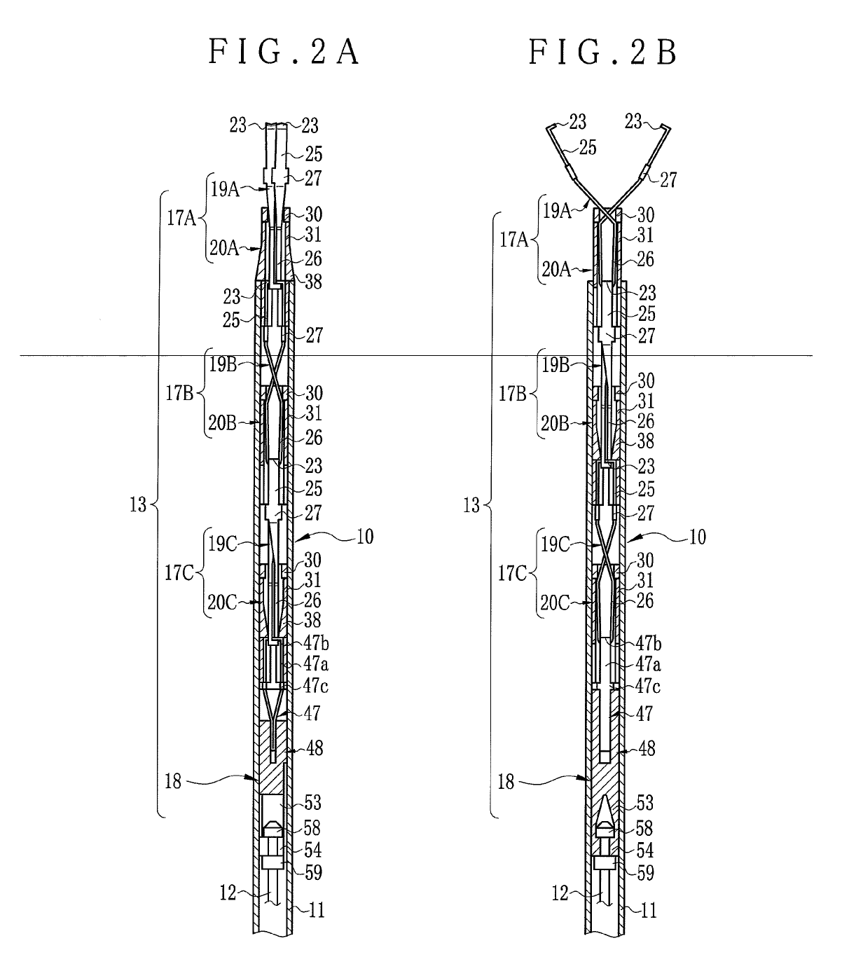

In FIG. 2A, the sheath end of the flexible sheath 11 loaded with the multiple clip assembly 13 is depicted for a state shortly before tissue clamping of a first one of the clip devices. In FIG. 2B, the sheath end is viewed with a difference of 90 degrees from FIG. 2A.

The multiple clip assembly 13 includes a train of hemostatic clip devices 17 and a fastening clip device 18. The hemostatic clip devices 17A, 17B and 17C (in place of the numeral 17) are fastened to one another serially. The fastening clip device 18 is fastened to the clip device 17C which is one of the clip devices 17 disposed on the proximal side. The clip devices 17 include clips 19 and tubular shells 20 or retaining rings. The tubular shells 20A, 20B and 20C (in place of the numeral 20) are disposed around respectively the clips 19A, 19B and 19C (in place of the numeral 19). Among the clips 19, a proximal end of a first one of the clips 19 is fastened to a second one of the clips 19 so as to fasten the clip devices 17 in the train.

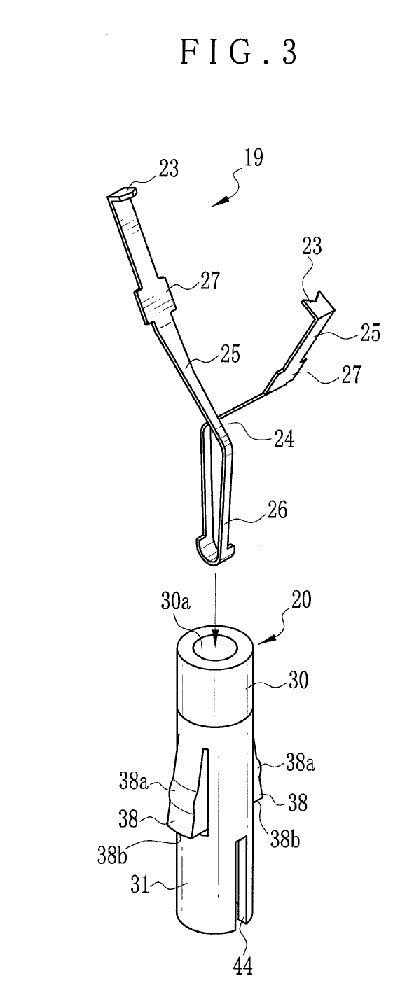

In FIG. 3, two claws 23 are formed with each of the clips 19. At first, an elongate strip of a single plate is bent at an angle of 180 degrees. Segment portions extending at ends of the strip are intersected with one another, and opposed to one another by curving, to define the claws 23. The clip 19 includes a crossed portion 24, arms 25 and a turn 26. The crossed portion 24 is defined by crossing the strip. The arms 25 are located at the free ends. The turn 26 is constituted by the closed end. Side projections 27 are formed on edges of the arms 25, are positioned at an intermediate point, and partially define portions with a greater width.

When the arms 25 of the clip 19 are free without receiving external force, the claws 23 are open and away from one another. The claws 23 become meshed with one another and are in a closed position for tissue clamping when the arms 25 are deformed to come near to one another. The claws 23 are a projection and a notch in the combination for the purpose of clamping tissue. An example of material for the clip 19 is metal with biocompatibility, for example stainless steel SUS 631 for springs.

In FIG. 3, the tubular shells 20 of a hollow shape receive insertion of the turn 26 of the clip 19 and set on the outside of the clip 19. The tubular shells 20 have an outer diameter approximately equal to an inner diameter of the flexible sheath 11, and are formed from a plastic material having flexibility, so as to move back and forth smoothly in the flexible sheath 11 even with a tortuous curve.

Each of the tubular shells 20 includes a push sleeve 30 and a support sleeve 31. The push sleeve 30 shifts the claws 23 to a closed position. The support sleeve 31 maintains a fastened state of the clips 19. The push sleeve 30 as a part of metal is fixed firmly on the support sleeve 31. A narrow bore 30a is defined in the push sleeve 30, greater than a width of the clip 19 near to the crossed portion 24, and smaller than the width of the area of the side projections 27. An example of material for the push sleeve 30 is metal with biocompatibility, for example stainless steel SUS 304.

When the tubular shell 20 is positioned around the clips 19, the push sleeve 30 is in an initial position to cover the crossed portion 24. The arms 25 are in an open position when the push sleeve 30 is in the initial position. When the push sleeve 30 shifts from the initial position to a position of contacting the side projections 27, the arms 25 are pushed by an edge of the narrow bore 30a to close the claws 23. The claws 23 exert force of clamping upon the push of the arms 25.

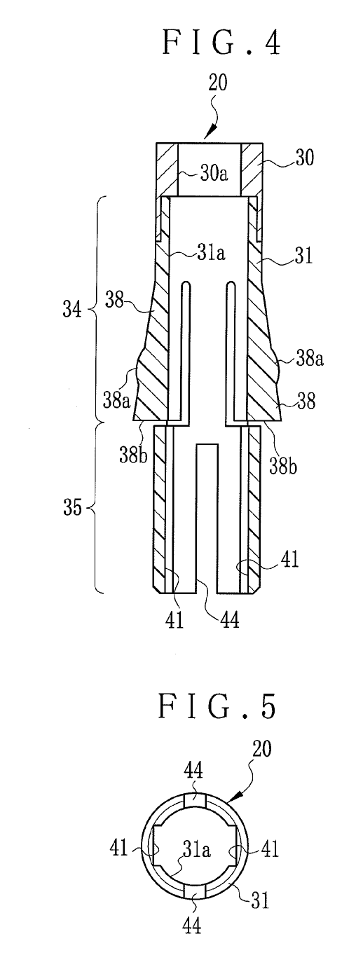

In FIG. 4, a bore 31a is defined in the support sleeve 31 of a cylindrical shape, and adapted to contain the turn 26 and the arms 25 of one of the clips 19. The support sleeve 31 includes a first region 34 of deployment, and a second region 35 of flexibility on a proximal side.

Fins 38 or skirt portions are disposed in the first region 34 and deploy radially from the outside of the support sleeve 31. A proximal fin end of the fins 38 extends from the support sleeve 31 resiliently. A distal fin end is partially separate from the support sleeve 31, and shiftable radially for deployment in a free state and for stowage upon depression. Two of the fins 38 are arranged about the axis of the support sleeve 31 symmetrically. Positions of the fins 38 according to the longitudinal direction of the support sleeve 31 are equal.

Projections 38a are formed to protrude from respectively the fins 38 in outward directions. The fins 38 have respectively fin ends 38b. The projections 38a operate by contact for closing the fins 38 to prevent interference of the fin ends 38b with the sheath end of the flexible sheath 11 in the course of insertion of the tubular shell 20 into the flexible sheath 11 by advancing the proximal end of the tubular shell 20. While the tubular shell 20 is disposed in the flexible sheath 11, the projections 38a contact the inside of the flexible sheath 11 for reducing friction.

In FIG. 5, two grooves 41 are formed in the bore 31a, located in the second region 35, opposed to one another, and positioned equally with the fins 38 in the rotational direction. The width of the grooves 41 is slightly greater than the maximum width of the arms 25 of the clip 19, and smaller than the width of the area of the side projections 27. A distance between wall surfaces of the grooves 41 is equal to a sum of lengths of the claws 23 of the clip 19 in the opening direction.

A size of a range of the second region 35 is equal to a distance of moving the push sleeve 30 from the initial position of the clip 19 toward its distal end to contact the side projections 27, namely a moving distance required for completely closing the clip 19. Also, the size of the range of the second region 35 is equal to a size of the clip 19 from the claws 23 to an upper end of the side projections 27.

Two end channels 44 are formed in the second region 35 at its end, and disposed with a difference from the fins 38 with an angle of 90 degrees. The end channels 44 make the tubular shell 20 flexible. The proximal end of the tubular shell 20 can open by operation of the end channels 44, to facilitate engagement of two of the clips 19 within the tubular shell 20. In view of this aspect, the support sleeve 31 is formed from a material which has biocompatibility and has sufficient resiliency and rigidity for the performance of the fins 38. A preferable example of the material for the support sleeve 31 is polyphenylsulfone (PPSU or PPS).

In the tubular shell 20A of FIGS. 2A and 2B, the first region 34 receives insertion of the turn 26 of the clip 19A through the narrow bore 30a. The clip 19A is rotationally set with a difference of the opening direction of the claws 23 from a deploying direction of the fins 38 at an angle of 90 degrees. The tubular shell 20A is mounted around the clip 19A to set the push sleeve 30 in the initial position of covering the crossed portion 24 of the clip 19A. In the clip 19A in the first region 34, a proximal end of the turn 26 protrudes into the second region 35 when in the initial position. The arms 25 protruding to the outside of the tubular shell 20A are open.

The arms 25 of the clip 19B are inserted in the second region 35 of the tubular shell 20A through the proximal end of the bore 31a. As the clip 19B is rotationally shifted with a difference of 90 degrees for an opening direction of the arms 25 from the clip 19A in the first region 34. The arms 25 are set in the grooves 41. The claws 23 of the clip 19B in the second region 35 are engaged with the turn 26 of the clip 19A. The tubular shell 20A operates to keep fastening between the clips 19A and 19B by maintaining the closed state of the claws 23 of the clip 19B in the second region 35 without shifting to the open state.

When the tubular shell 20 is contained in the flexible sheath 11, the fins 38 are depressed and stowed by the flexible sheath 11. See the tubular shell 20B of FIG. 2B. Inner surfaces of the fins 38 clamp sides of the turn 26 of the clip 19B. Thus, the clip 19B can be firmly fastened to the tubular shell 20B. No offsetting occurs between the clips 19 and the tubular shells 20 in a rotational direction and axial direction.

In the clip 19C inserted in the second region 35 of the tubular shell 20B, the arms 25 become engaged with the grooves 41. The claws 23 become engaged with the clip 19B located in the first region 34. The side projections 27 contact a proximal end of the tubular shell 20B. Thus, the clip 19C is prevented from rotating and moving back or forth relative to the tubular shell 20B. The multiple clip assembly 13 in the flexible sheath 11 can move back and forth and rotate in the flexible sheath 11 without differences between the clips 19 and the tubular shell 20.

See the tubular shell 20A in FIG. 2B. When the tubular shell 20 is pushed out of the sheath end of the flexible sheath 11, the fins 38 come to open outwards by their resiliency, and extend with a greater width than the bore of the flexible sheath 11, to prevent the tubular shell 20A from moving backwards in the flexible sheath 11. The clip 19A in the first region 34 is released from clamping of the fins 38, and becomes movable relative to the tubular shell 20A. Then the operating wire 12 is pulled to move back the clip 19A. The tubular shell 20A moves forwards relative to the clip 19A, and closes the clip 19A.

The second region 35 of the tubular shell 20A keeps the fastened state of the clips 19A and 19B while the clip 19A in the first region 34 moves back relative to the tubular shell 20A and becomes closed, so that force of pull of the clip 19B is transmitted to the clip 19A. When the clip 19A of the first region 34 is closed completely, the turn 26 of the clip 19A and the clip 19B engaged with the turn 26 move out of the tubular shell 20A. Thus, the claws 23 are open by their resiliency to disengage the clip 19B from the clip 19A.

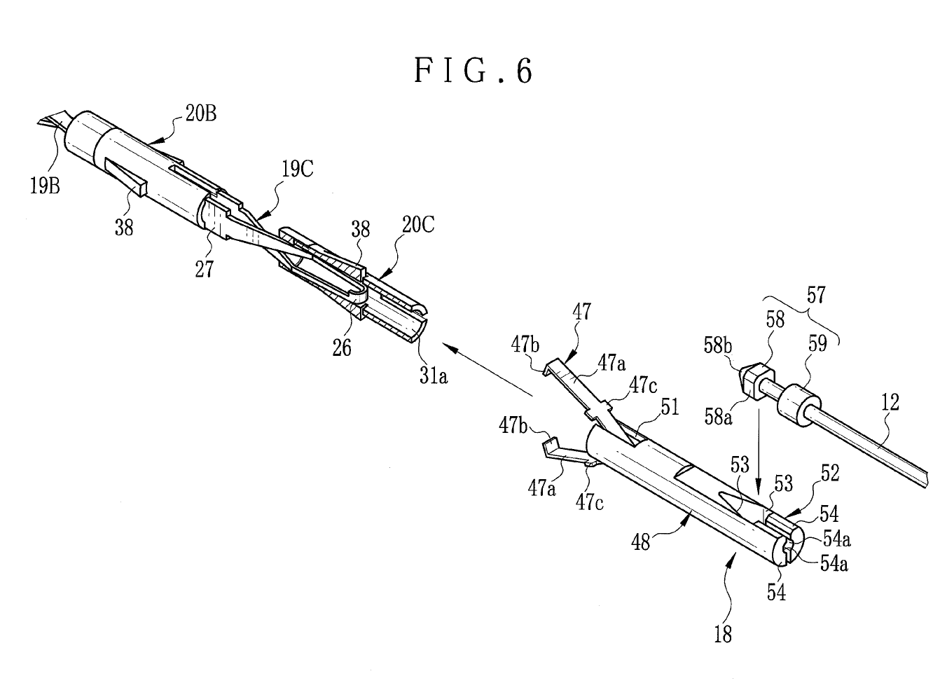

In FIGS. 2A and 2B, the fastening clip device 18 not for the tissue clamping is fastened to the clip 19C. In FIG. 6, the fastening clip device 18 includes a fastening clip 47 or dummy clip and a support 48. A pair of arms 47a of the fastening clip 47 are defined by bending a single elongate strip of a metal plate. The arms 47a are in an open state if free without external force. Jaws 47b are formed with ends of the arms 47a. Side projections 47c are formed with intermediate portions of the arms 47a. The fastening clip 47 may be produced from a material the same as that for the clips 19.

The fastening clip 47 becomes inserted in the bore 31a of the tubular shell 20C by rotationally changing the direction of the arms 47a with a difference of 90 degrees from the direction of opening and closing of the clip 19C on the proximal side. The jaws 47b of the fastening clip 47 are engaged with the turn 26 of the clip 19C. The tubular shell 20C is kept in connection by the second region 35 as the jaws 47b are kept from moving to the open position.

The support 48 is a cylindrical part produced from a material the same as the support sleeve 31 of the tubular shell 20. A support recess 51 is formed in the support 48 on the distal side, and supports the fastening clip 47. A fastening mechanism 52 is disposed on the proximal side of the support 48, and adapted to fastening to the operating wire 12.

The fastening mechanism 52 includes a pair of cavity walls 53 with resiliency, and a pair of clamping walls 54. The cavity walls 53 are resilient in the radial direction of the support 48. The clamping walls 54 are located at the end of the cavity walls 53. An interval between the clamping walls 54 is smaller than an outer diameter of the operating wire 12. Also, grooves 54a of an arcuate shape as viewed in section are formed inside the clamping walls 54, extend along the axis of the support 48, and have an equal diameter of the operating wire 12.

In FIGS. 2A and 2B, the claws 23 of the clip 19B in the multiple clip assembly 13 are engaged with the turn 26 of the clip 19A. The engaged portion is surrounded by the tubular shell 20A. The inside of the second region 35 of the tubular shell 20A keeps the claws 23 closed in the clip 19B. This maintains the fastened state between the clips 19A and 19B. Similarly, the clip 19C is kept fastened to the clip 19B by the tubular shell 20B. The fastening clip device 18 is kept fastened to the clip 19C by the tubular shell 20C.

An example of the flexible sheath 11 is a flexible coil sheath in which a wire of metal is tightly wound in a coiled form. An inner diameter of the flexible sheath 11 is so determined that the turn 26 of a first one of the clips 19 is disengaged from the claws 23 of a second one of the clips 19. The inner diameter of the flexible sheath 11 is greater than a sum of a length of the claws 23 and a width of an engaged portion of the turn 26 with the claws 23.

The operating wire 12 is a wire of metal having biocompatibility. In FIG. 6, a shaft head 57 for hooking is disposed at the wire end of the operating wire 12 for connection with the fastening clip device 18. The shaft head 57 includes a front shaft head portion 58 and a rear shaft head portion 59 arranged on the operating wire 12.

The front shaft head portion 58 includes a lateral surface 58a on a quadrilateral prismatic part, and an inclined surface 58b on a quadrilateral pyramidal part. The lateral surface 58a has one side line which has a length equal to the size of the clearance between the cavity walls 53. The inclined surface 58b has a size corresponding to the clearance between the cavity walls 53. The rear shaft head portion 59 has a cylindrical shape having a diameter which is greater than the outer diameter of the front shaft head portion 58 and slightly smaller than an outer diameter of the support 48. The rear shaft head portion 59 is distant from a proximal end of the front shaft head portion 58 by a distance which is equal to the length of the clamping walls 54 in the axial direction.

The front shaft head portion 58 is inserted between the cavity walls 53 by moving downwards. Similarly, a portion of the operating wire 12 between the shaft head portions 58 and 59 is inserted between the clamping walls 54 by moving downwards, and clamped by the grooves 54a. The lateral surface 58a of the front shaft head portion 58 contacts the cavity walls 53. A proximal end of the lateral surface 58a contacts a distal end of the clamping walls 54. A distal end of the rear shaft head portion 59 contacts a proximal end of the clamping walls 54.

A surface of the proximal end of the front shaft head portion 58 presses the distal end of the clamping walls 54 when the operating wire 12 is pulled, to transmit the force to the multiple clip assembly 13. When the operating wire 12 rotates, the lateral surface 58a of the front shaft head portion 58 transmits rotation to the cavity walls 53, so that the multiple clip assembly 13 rotates together. Also, when the flexible sheath 11 is pulled (or moved back relative to the operating wire 12), the distal end of the rear shaft head portion 59 contacts the proximal end of the clamping walls 54. This prevents the multiple clip assembly 13 from moving together with the flexible sheath 11.

In FIG. 1, the handle assembly 14 includes a wire handle 62 and a sheath handle 63. The wire handle 62 is cylindrical. An elongated pipe 64 is disposed at a distal end of the wire handle 62, and has a smaller diameter than the wire handle 62. An opening 65 is formed in the middle of the wire handle 62. There is a pull arm 66 of which a rear portion appears partially through the wire handle 62. An operator inserts his or her finger to pull the pull arm 66. A proximal end of the operating wire 12 is retained on the pull arm 66, the operating wire 12 extending through the sheath handle 63, the elongated pipe 64 and the wire handle 62 by insertion.

The pull arm 66 is kept movable back and forth in the wire handle 62, namely in an axial direction of the wire handle 62. The pull arm 66, when in a home position, appears in the opening 65, and when in a pull position, is slid in the axial direction. A length of slide of the pull arm 66 is determined to correspond to a length of pull of the operating wire 12 within the flexible sheath 11 for closing the clips 19 protruding from the sheath end of the flexible sheath 11. A spring (not shown) biases the pull arm 66 toward the home position. The spring compresses when the pull arm 66 is pulled back, but pushes the pull arm 66 forwards when the pull arm 66 is released from the finger.

The sheath handle 63 has a cylindrical shape and has an opening at its proximal end. A proximal end of the flexible sheath 11 is attached to a distal end of the sheath handle 63. When the sheath handle 63 is pulled back toward the wire handle 62, the flexible sheath 11 is pulled. The sheath handle 63 is mounted around the elongated pipe 64 in a manner slidable in the axial direction of pulling the flexible sheath 11.

A lock mechanism is disposed in the sheath handle 63. Notches 69 are formed on an upper portion of the elongated pipe 64, and adapted to locking the sheath handle 63 in one of plural positions of slide. A release button 70 is disposed on an upper portion of the sheath handle 63, and operable for releasing the lock mechanism for sliding. An interval between the notches 69 corresponds to a length of moving the flexible sheath 11 for each time of advancing one of the clip devices 17 from the flexible sheath 11. The number of the notches 69 corresponds to the number of the clip devices 17 insertable in the flexible sheath 11.

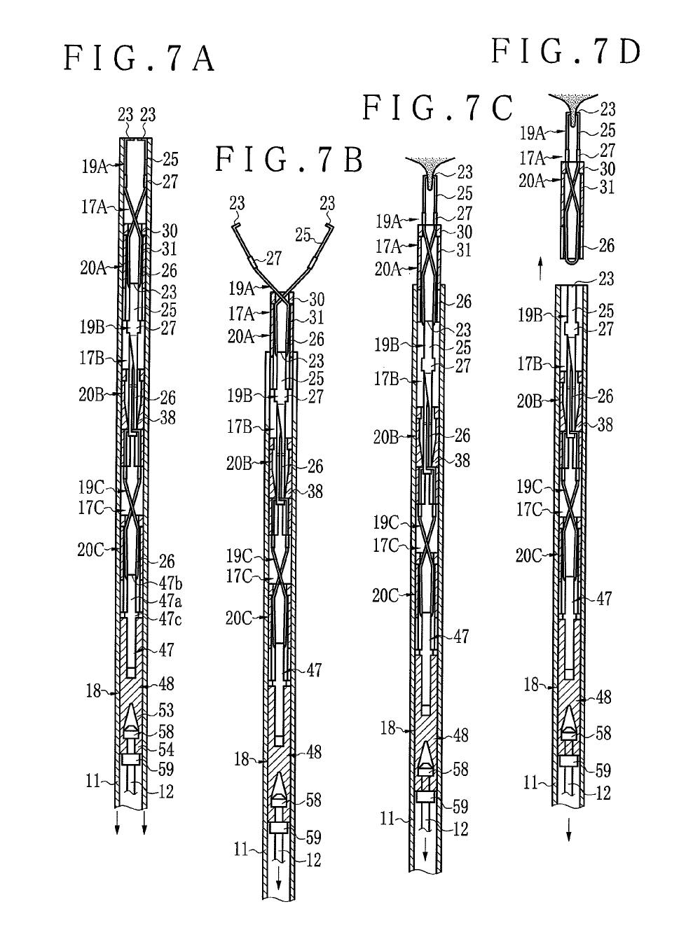

The operation of the multiple hemostatic clip application apparatus 10 is described now. In FIG. 7A, the flexible sheath 11 is loaded with the multiple clip assembly 13 including three of the clip devices 17 and the fastening clip device 18. Shortly after loading with the multiple clip assembly 13, a distal end of the first clip 19A is flush with a sheath end of the flexible sheath 11. The claws 23 are kept in the closed position by the inside of the flexible sheath 11.

The flexible sheath 11 is inserted in a forceps channel of the endoscope entered in a body cavity. The sheath end of the flexible sheath 11 protrudes from the forceps opening of the endoscope, and accesses a lesion. In this state, the sheath handle 63 in the handle device 14 is pulled to shift an engaging claw from the first to the second of the notches 69. Thus, the operating wire 12 does not move in contrast with the flexible sheath 11.

In FIG. 7B, when the flexible sheath 11 is pulled at a predetermined distance equal to a distance between the first and second of the notches 69, then the sheath end comes back to a position to deploy the fins 38 of the tubular shell 20A at the distal end. The claws 23 of the clip 19A protruding from the flexible sheath 11 are deployed with resiliency to the deployed position. Thus, the clip 19A becomes ready for use.

When the flexible sheath 11 is pulled, force of friction is exerted between the flexible sheath 11 and the tubular shells 20A-20C inside. However, the tubular shells 20A-20C in connection are kept without deviation rotationally or back and forth, because tightly engaged with the clips 19A-19C by the fins 38. An area of contact and force of friction of the tubular shells 20 are small, because of the contact with the inside of the flexible sheath 11 by the projections 38a of the fins 38. Thus, the tubular shells 20A-20C do not move relative to the clips 19A-19C incidentally even if the flexible sheath 11 is pulled. The tubular shells 20A-20C can be maintained in the state of retaining the clips 19A-19C.

Then the multiple hemostatic clip application apparatus 10 is moved, to apply pressure of the claws 23 of the clip 19A in the open position to tissue of a body part to be clamped. The pull arm 66 of the wire handle 62 is pulled. The operating wire 12 is pulled at a predetermined length, to pull together the clips 19A-19C engaged in the sequence from the fastening clip device 18.

In the state of FIGS. 7B and 7C, the fins 38 of the tubular shell 20A protruding from the flexible sheath 11 are deployed. The clip 19A is free from retention with the fins 38. The tubular shell 20A is prevented from returning into the flexible sheath 11 by the fins 38, which are kept deployed by the sheath end. In FIG. 7C, the first clip 19A is moved back relative to the tubular shell 20A by the pull of the operating wire 12. Thus, the push sleeve 30 is positioned directly under the side projections 27 of the clip 19A, finally to close the clip 19A by the tubular shell 20A.

At the same time as the clip 19A is closed fully, the engaged portion between the clips 19A and 19B comes away from the proximal end of the tubular shell 20A. The arms 25 of the clip 19B are open with their resiliency to contact the inside of the flexible sheath 11. An interval between the claws 23 becomes greater than the width of the turn 26 of the clip 19A to disengage the clip 19A from the clip 19B. In FIG. 7D, the entirety of the multiple hemostatic clip application apparatus 10 is moved to release the sheath end of the flexible sheath 11 from the tissue or lesion of a body part, to separate the clip device 17A from the sheath end.

The pull arm 66 is returned by the bias of the spring to the home position upon termination of its pull. Thus, the operating wire 12 moves toward the distal end inside the flexible sheath 11 to push the fastening clip device 18 and the clips 19B and 19C. A distal end of the clip 19B becomes flush again with the sheath end of the flexible sheath 11 as illustrated in FIG. 7D. In a manner similar to the first clip device 17A, the clip devices 17B and 17C are operated for tissue clamping by manual handling of the handle device 14.

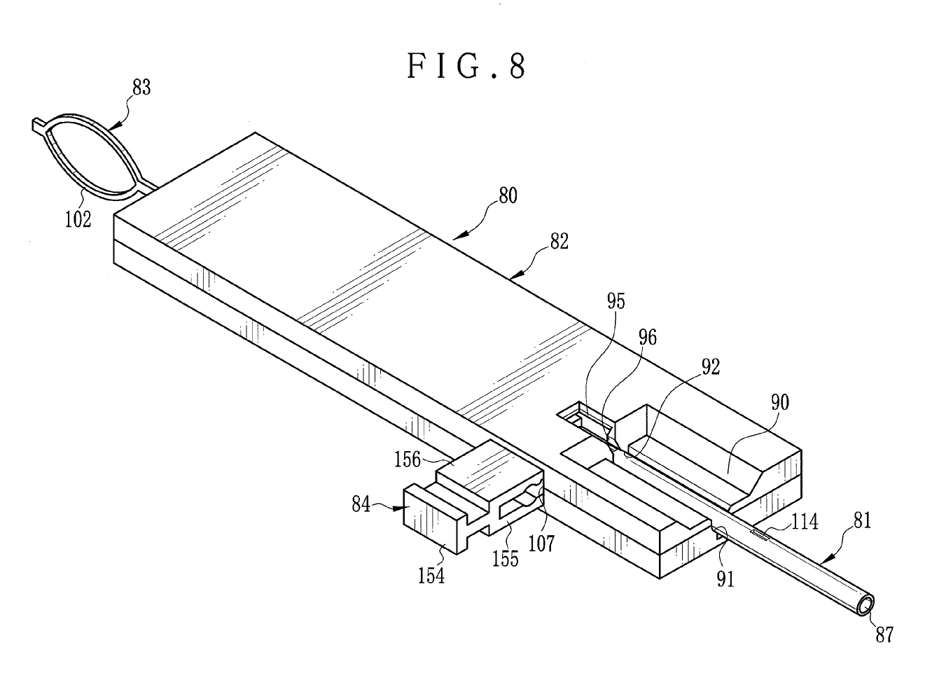

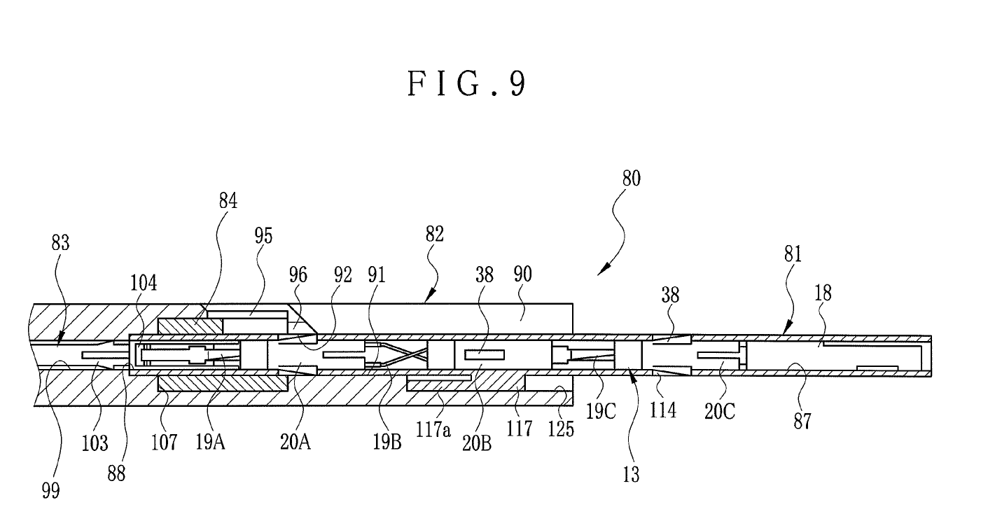

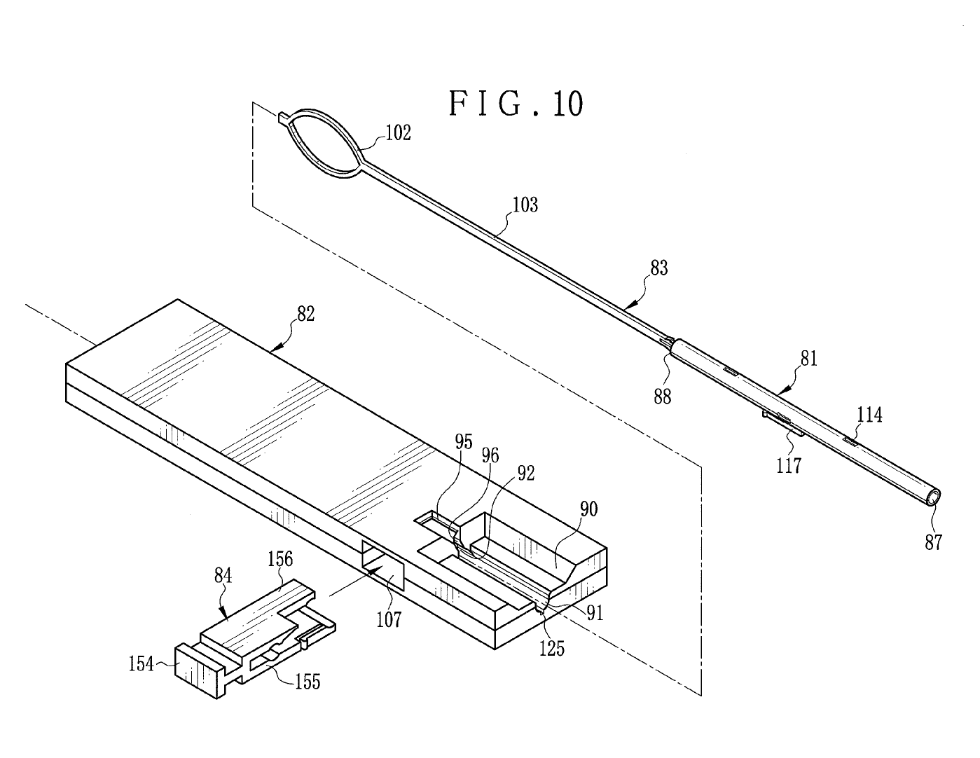

A multiple clip package of the invention is described now. In FIGS. 8-10, a multiple clip package 80 or multiple clip holder includes a housing 81 or barrel and a coupling device 82. The housing 81 contains the multiple clip assembly 13. The coupling device 82 has a box shape, and receives insertion of an end of the housing 81. The coupling device 82 includes a pull rod structure 83 and a slider 84. The pull rod structure 83 as a clip moving structure enters the multiple clip assembly 13 into the coupling device 82 from the housing 81. The slider 84 is a guide mechanism operable for fastening the operating wire 12 to the fastening clip device 18.

A barrel cavity 87 is defined in the housing 81. An outer diameter of the housing 81 is substantially equal to an outer diameter of the flexible sheath 11. A diameter of the barrel cavity 87 is substantially equal to the inner diameter of the flexible sheath 11. The multiple clip assembly 13 is contained in the barrel cavity 87. An exit opening 88 is open at a distal end of the housing 81. The clip 19A in the multiple clip assembly 13 is disposed close to the exit opening 88 on the distal side of the housing 81.

The coupling device 82 operates as a body of the multiple clip package 80, depresses the fins 38 of the multiple clip assembly 13 introduced from the housing 81, and loads the flexible sheath 11 with the multiple clip assembly 13. A stage groove 91 or housing receiving groove is formed in the coupling device 82, is disposed at its end as viewed in the longitudinal direction, has an inner diameter slightly greater than an outer diameter of the housing 81, and has an open upper side. Also, a recess 90 is formed in a peripheral portion of the stage groove 91, and causes a portion of the housing 81 to appear through the stage groove 91. An access hole 92 is formed in a wall of the recess 90 and communicates with the stage groove 91.

The housing 81 is inserted in the stage groove 91 and the access hole 92. For the purpose of loading the flexible sheath 11 with the multiple clip assembly 13 from the coupling device 82, the housing 81 is removed from the stage groove 91 and the access hole 92 before inserting the flexible sheath 11 instead. The flexible sheath 11 or the housing 81 inserted in the stage groove 91 appears partially upwards. It is possible to retain the housing 81 and the coupling device 82 or the flexible sheath 11 and the coupling device 82 together by holding in the recess 90.

A connection opening 95 and a wire channel 96 are formed in the upper wall of the coupling device 82 in connection with the recess 90. The connection opening 95 is open for insertion of the shaft head 57 of the operating wire 12 into the coupling device 82 in a direction transverse to a direction in which the housing 81 is inserted in the coupling device 82. An open area of the connection opening 95 is larger than an area of the shaft head 57 as viewed laterally. The wire channel 96 extends for connecting the access hole 92 with the connection opening 95, and has such a dimension as to receive insertion of the operating wire 12 laterally. When the flexible sheath 11 is inserted in the stage groove 91, the operating wire 12 and the shaft head 57 are inserted in the wire channel 96 and the connection opening 95 simultaneously, and become connected with the support 48 of the fastening clip device 18 introduced in the coupling device 82. See FIG. 6.

A fin bending channel 99 or skirt bending channel is formed in the coupling device 82, is positioned inwards from the access hole 92, and has a cylindrical form. The fin bending channel 99 has a diameter equal to an inner diameter of the flexible sheath 11 and that of the barrel cavity 87, and comes in registration with the flexible sheath 11 and the housing 81 inserted in the access hole 92. The fin bending channel 99 internally depresses and stows the fins 38 of the multiple clip assembly 13 introduced through the barrel cavity 87.

The pull rod structure 83 includes a pull tab portion 102, a shank 103 and an end connector 104. The pull tab portion 102 protrudes from the coupling device 82 in its longitudinal direction, and has an elliptic shape. The shank 103 extends from the pull tab portion 102. The end connector 104 is formed with an end of the shank 103. The shank 103 has a length enough to penetrate the fin bending channel 99, and is inserted in the same. The end connector 104 is inserted in the barrel cavity 87, and engaged with the first clip 19A.

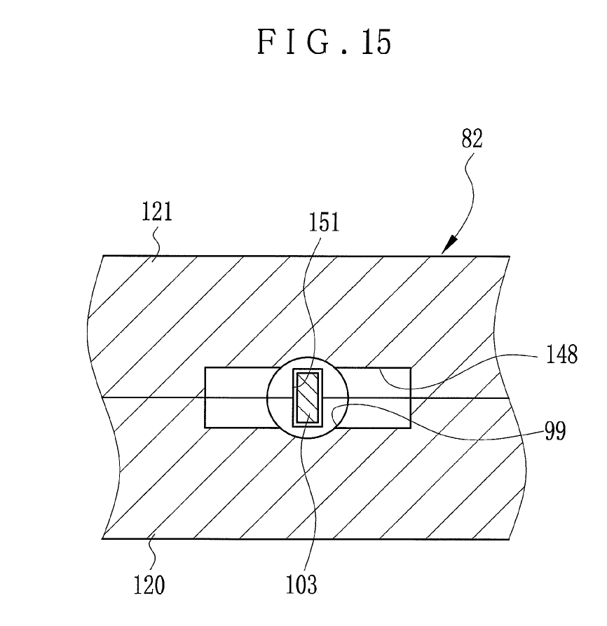

The pull rod structure 83 is deformed resiliently to depress the annular shape of the pull tab portion 102 and becomes inserted in the access hole 92 together with the housing 81. The shank 103 is entered in the fin bending channel 99. The pull tab portion 102 comes to protrude to the outside of the coupling device 82 from a pull opening at the end of the fin bending channel 99. See the reference numeral 151 in FIG. 15. When the pull tab portion 102 is pulled relative to the coupling device 82, the first clip 19A of the multiple clip assembly 13 is pulled by the end connector 104 and entered in the fin bending channel 99 through the barrel cavity 87.

A slide channel 107 is formed through a lateral wall of the coupling device 82, and receives insertion of the slider 84 in a slidable manner. The slider 84 operates when pressed in the slide channel 107 for connecting the fastening clip device 18 introduced in the coupling device 82 with the shaft head 57 inserted through the connection opening 95.

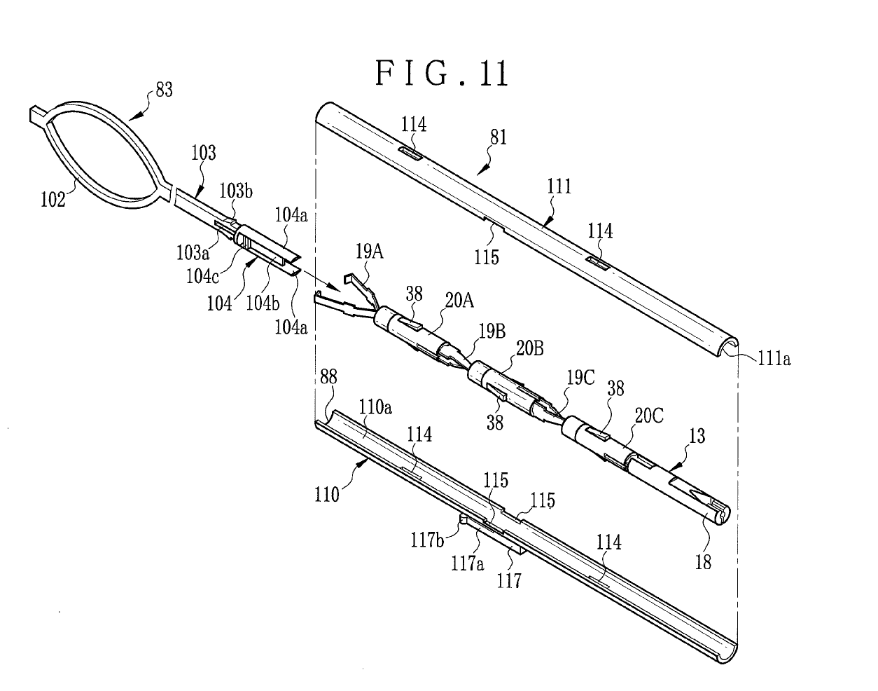

In FIG. 11, the housing 81 is constituted by a lower housing half 110 and an upper housing half 111 or barrel halves. An inner surface 110a of the lower housing half 110 and an inner surface 111a of the upper housing half 111 are semicylindrical, and combined to form the barrel cavity 87 of the cylindrical shape. The barrel cavity 87 is open at both of two ends, which include a first end for containing the first clip 19A and a second end for containing the fastening clip device 18. The exit opening 88 at the first end is open for advancing the multiple clip assembly 13 to the outside of the barrel cavity 87.

The multiple clip device 13 is contained in the barrel cavity 87 while the claws 23 of the first clip 19A are positioned laterally in the horizontal direction. The first clip 19A is set in the closed position by the inside of the barrel cavity 87. As has been described heretofore, the clip devices 17A-17C are oriented with differences in the opening direction of the claws 23 with 90 degrees from one another. There is a difference between the claws 23 and the fins 38 in the opening direction with 90 degrees. Thus, the fins 38 of the tubular shells 20A and 20C are movable for deployment vertically in the depicted state. The fins 38 of the tubular shell 20B are movable for deployment horizontally in the depicted state.

Fin receiving slots 114 or skirt receiving slots are formed in the upper and lower housing halves 110 and 111 and receive the fins 38 deployed from the tubular shells 20A and 20C. Recesses are formed in edge portions of the upper and lower housing halves 110 and 111, and define fin receiving slots 115 or skirt receiving slots when joined, so as to receive the fins 38 deployed from the tubular shell 20B. This is effective in setting the fins 38 free from the pressing force toward the stowed position inside the housing 81 in the course of preservation. The force of recovery of the fins 38 toward the deployed position can be kept without lowering. Note that recesses or grooves may be formed in place of through holes or the fin receiving slots 114 and 115.

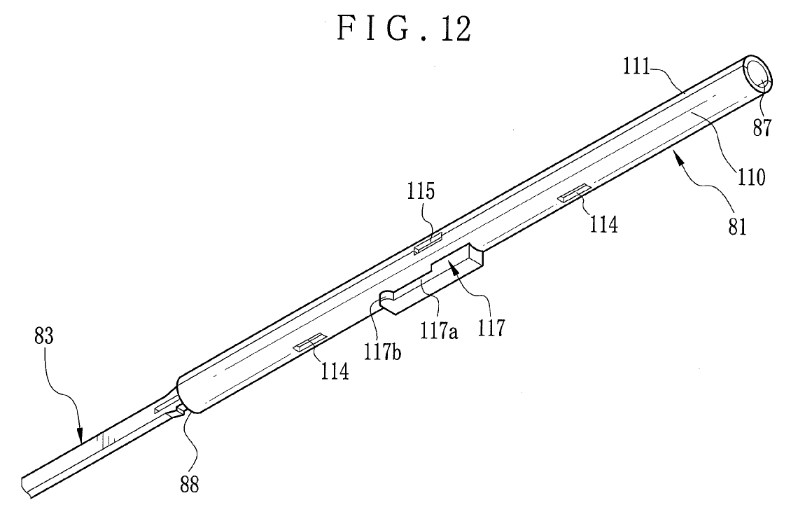

In FIG. 12, a key projection 117 projects from a lower portion of the lower housing half 110, and is positioned in the middle of the housing 81 in the axial direction. The key projection 117 is included in one piece of the lower housing half 110. A spring arm 117a is included in the key projection 117, is positioned on a lower side, and extends in an axial direction of the housing 81. A proximal end of the spring arm 117a is associated with the proximal end of the housing 81. A distal end of the spring arm 117a is a free end. The spring arm 117a is deformable resiliently transversely to the axial direction of the housing 81. A retaining hook 117b of a curved shape projects from a lateral surface of the spring arm 117a.

The housing 81 is formed from a transparent plastic material for an operator externally to view the multiple clip assembly 13 inside the barrel cavity 87. To join the upper housing half 111 with the lower housing half 110, it is possible to use adhesive agent for adhesion, ultrasonic waves for welding, claws for engagement and the like. Also, transparent plastic film can be wound on the periphery of the housing obtained by combining the housing halves 110 and 111.

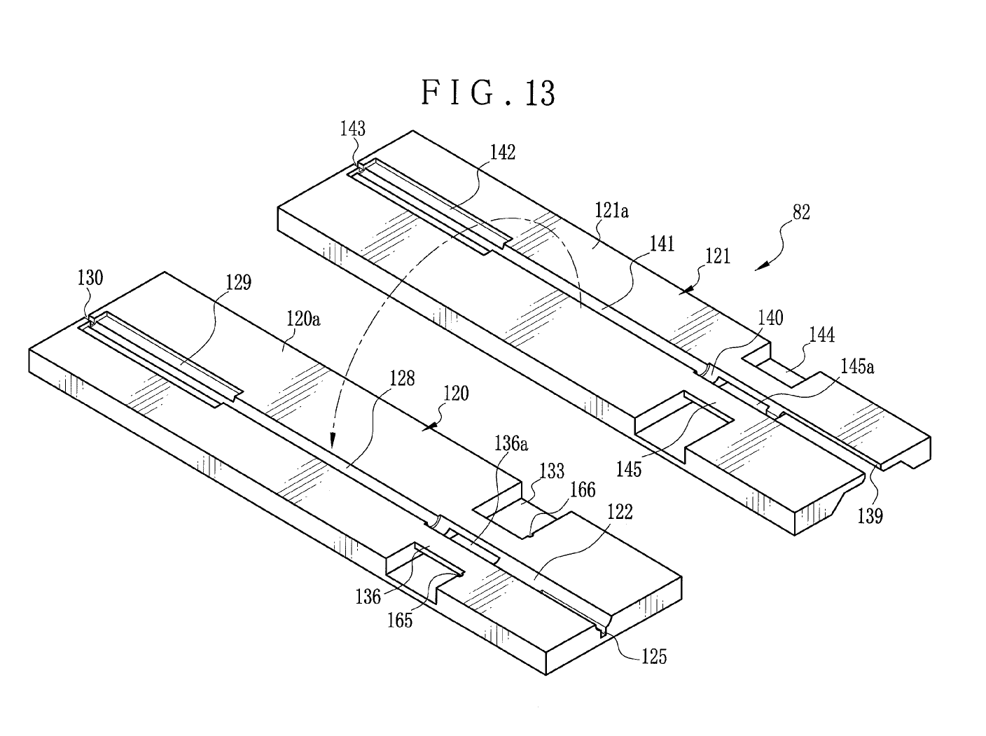

In FIG. 13, the coupling device 82 is constituted by a lower casing 120 and an upper casing 121. An upper surface 120a is defined by the upside of the lower casing 120. An elongated recess 122 is formed in the upper surface 120a, and defines the stage groove 91 and the access hole 92 upon joining the upper casing 121.

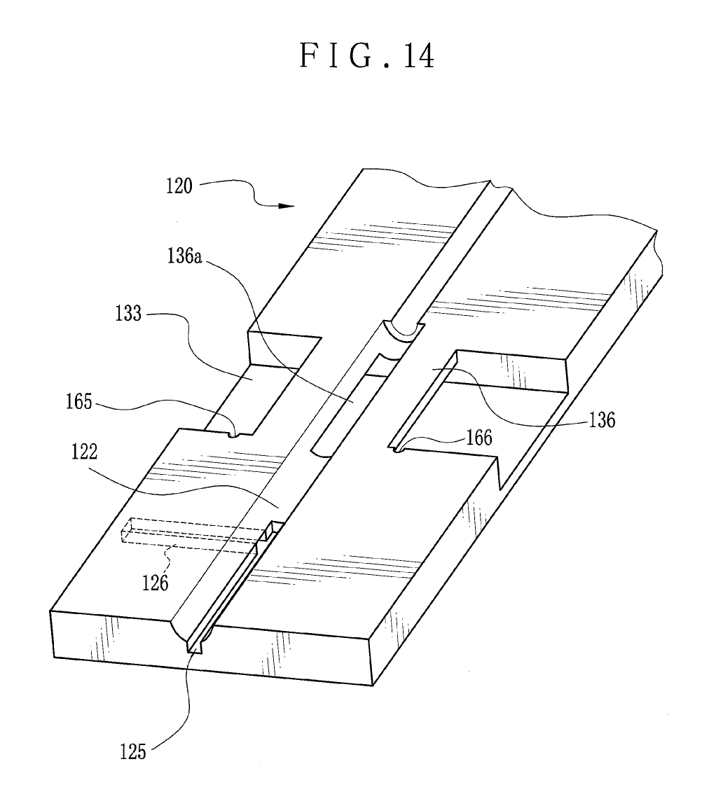

In FIG. 14, a key way groove 125 is formed in the surface of the elongated recess 122. When the housing 81 is inserted in the stage groove 91 and the access hole 92 by advance in the axial direction, the key way groove 125 is engaged with the key projection 117 on the housing 81 for blocking rotation. The engagement of the key projection 117 with the key way groove 125 prevents rotation of the housing 81, and sets the housing 81 in a suitable orientation in the coupling device 82.

A retaining opening 126 of click is formed in a lateral surface of the key way groove 125, and has a quadrilateral shape as viewed in a section. The retaining opening 126 is positioned to correspond to the retaining hook 117b of the key projection 117. When the key projection 117 is inserted in the key way groove 125 to a predetermined position, the retaining hook 117b is moved into and engaged with the retaining opening 126. Thus, the retaining hook 117b and the retaining opening 126 operate to prevent separation of the housing 81 from the coupling device 82 by regulating the housing 81 in the axial direction. Also, the engagement of the retaining hook 117b with the retaining opening 126 with a click can notify a user of the status after insertion of the housing 81 to a predetermined position through the access hole 92. In FIG. 14, the retaining opening 126 is formed through to extend to a lateral face of the lower casing 120. However, the retaining opening 126 can have a shape engageable with the retaining hook 117b, or can be an engageable recess.

A fin bending recess 128 or skirt bending recess is formed in the lower casing 120, extends from an end of the elongated recess 122, has a semicylindrical shape, and defines the fin bending channel 99 when the upper casing 121 is joined. A wall channel 129 for release is formed in an end portion of the lower casing 120, and extends with a greater width from the fin bending recess 128. A rectangular end channel 130 is disposed at an end of the fin bending recess 128, and receives insertion of the shank 103 of the pull rod structure 83.

A through opening 133 for slide is formed in a lower portion of the elongated recess 122, and constitutes the slide channel 107 when the upper casing 121 is joined. The through opening 133 has a quadrilateral shape, has a width equal to a width of the slider 84, and comes through the lower casing 120 transversely to its longitudinal direction.

In the coupling device 82, a bridge portion 136 extends between edge portions of the through opening 133 and is formed in a plate shape. The bridge portion 136 operates for reinforcement with strength at the through opening 133 in the coupling device 82, and is a stopper adapted upon sliding of the slider 84 into the slide channel 107. The elongated recess 122 is formed to extend through a part of the bridge portion 136. An access opening 136a is formed through the elongated recess 122 and communicates with the through opening 133.

The upper casing 121 has a lower surface 121a . An elongated slot 139 and an intermediate recess 140 are formed in the lower surface 121a. The elongated slot 139 constitutes the stage groove 91. The intermediate recess 140 has an arcuate shape and constitutes the access hole 92. Also, there are a fin bending recess 141 or skirt bending recess, a wall channel 142 for release, and an end channel 143 formed in the lower surface 121a. The fin bending recess 141 constitutes the fin bending channel 99. The end channel 143 is shaped similarly to the end channel 130. The upper casing 121 has a through opening 144 for slide, a bridge portion 145 and an access opening 145a. The through opening 144 constitutes the slide channel 107 near to the intermediate recess 140. The access opening 145a is formed to extend to the connection opening 95.

In FIG. 15, a release groove 148 is defined by a combination of the wall channels 129 and 142, and has a greater width horizontally than the fin bending channel 99 upon joining the upper and lower casings 120 and 121. A vertical size of the release groove 148 is greater than a width of the area of the side projections 27 of the clips 19. The horizontal width of the release groove 148 is sufficient for allowing the arms 25 of the clip 19A to open for disengagement from the end connector 104 of the pull rod structure 83. The release groove 148 is so positioned that, when the fastening mechanism 52 of the fastening clip device 18 becomes positioned at the access opening 145a, the clip 19A reaches the release groove 148.

The coupling device 82 is formed from a transparent plastic material for an operator externally to view the multiple clip assembly 13, the housing 81, the pull rod structure 83 and the slider 84. To join the upper casing 121 with the lower casing 120, it is possible to use adhesive agent for adhesion, ultrasonic waves for welding, claws for engagement and the like.

In FIG. 15, a pull opening 151 is defined by the end channels 130 and 143 in combination. The shank 103 of the pull rod structure 83 is inserted in the pull opening 151. A shape of the section of the shank 103 is similar to that of the pull opening 151. The shank 103 will not rotate in the fin bending channel 99 even if distortion occurs in the shank 103 during pull of the pull tab portion 102. Thus, the multiple clip assembly 13 can be introduced in the fin bending channel 99 constantly in the same rotational orientation as the state contained in the housing 81.

In FIG. 11, a center channel 103a is formed in the shank 103 of the end connector 104 and disposed near to its end. An anti-reverse projection 103b is formed to project from each of positions higher and lower than the center channel 103a, and extends with an increasing width from the pull tab portion 102 toward the end connector 104. The anti-reverse projection 103b extends to the outside of the coupling device 82 through the pull opening 151 by deformation of the shank 103 at the center channel 103a with resiliency. The anti-reverse projection 103b retains the shank 103 to prevent its return into the fin bending channel 99.

The end connector 104 has an outer diameter equal to that of the tubular shell 20. A pair of guide projections 104a are included in the end connector 104. A wall 104b in the end connector 104 is disposed between the guide projections 104a for keeping their interval. Engageable projections 104c project from lateral surfaces of the wall 104b, and are disposed near to the shank 103.

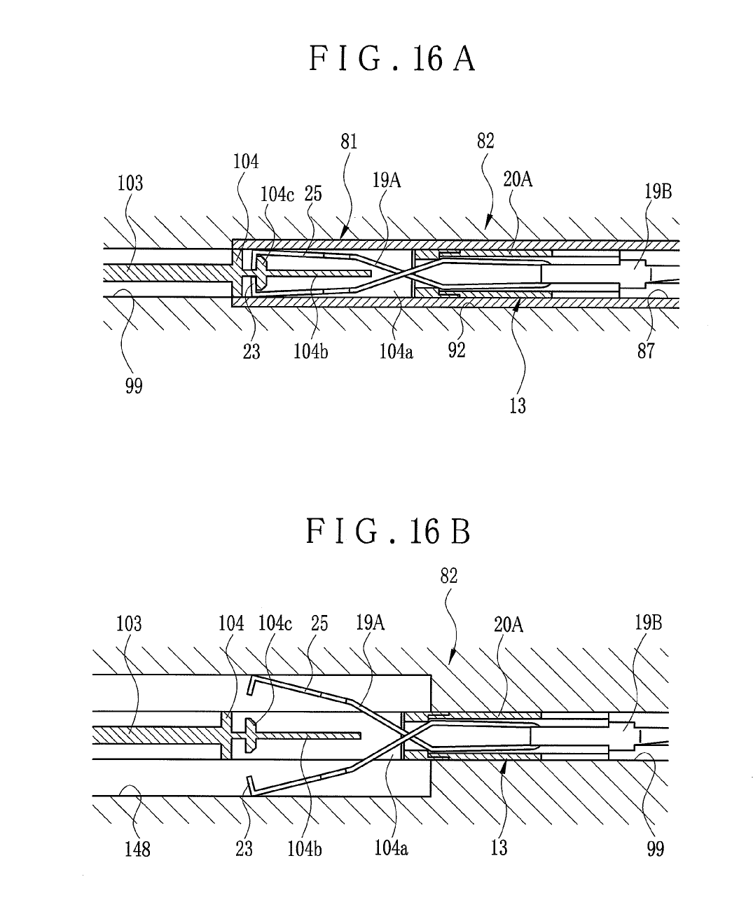

In FIG. 16A, the end connector 104 is contained in the barrel cavity 87 in the housing 81 with the multiple clip assembly 13. The clip 19A is kept closed by pressure of the barrel cavity 87. The claws 23 are engaged with the engageable projections 104c. When the pull tab portion 102 is pulled relative to the coupling device 82, the shank 103 slides in the fin bending channel 99 to move the end connector 104 from the barrel cavity 87 into the fin bending channel 99.

In FIG. 16B, the multiple clip assembly 13 is pulled by the end connector 104 and introduced in the fin bending channel 99. The first clip 19A shifts to the open position with its resiliency upon the reach to the release groove 148, and becomes disengaged from the end connector 104. Thus, the multiple clip assembly 13 is positioned within the fin bending channel 99 to oppose the fastening mechanism 52 of the fastening clip device 18 to the access opening 145a in the access hole 92.

In the clip devices 17, an opening direction of the claws 23 is different from a deploying direction of the fins 38 with a difference of 90 degrees. Even when the clip 19A is positioned at the release groove 148, the fins 38 of the tubular shell 20A do not open.

The pull rod structure 83 is one piece molded from plastic material having suitable resiliency, inclusive of the pull tab portion 102, the shank 103 and the end connector 104. Note that the end connector 104 may be a part separate from the shank 103, and can be joined with the shank 103 inside the coupling device 82.

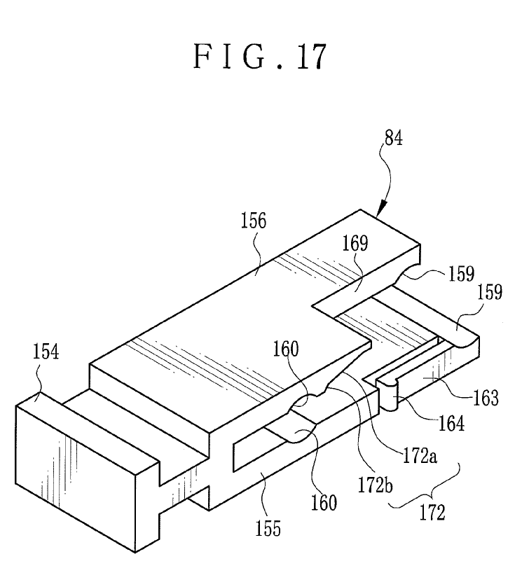

In FIG. 17, the slider 84 is one piece molded from a plastic material, and includes a button head 154, a lower slide plate 155 and an upper slide plate 156. The button head 154 is operable for depression into the slide channel 107. The slide plates 155 and 156 are opposed to one another, and extend from the button head 154 horizontally. The slide plates 155 and 156 are inserted in the through openings 133 and 144 of the upper and lower casings 120 and 121, and squeeze the bridge portions 136 and 145.

First receiving recesses 159 are formed in inner surfaces of the lower and upper slide plates 155 and 156. Second receiving recesses 160 are also formed and disposed in parallel with the first receiving recesses 159. The first and second receiving recesses 159 and 160 have inner diameters substantially equal to respectively the outer diameters of the flexible sheath 11 and the housing 81. The slider 84 is kept slidable between an initial position and a connecting position, and when in the initial position, registers the first receiving recesses 159 with the access hole 92, and when in the connecting position, registers the second receiving recesses 160 with the access hole 92. When the slider 84 comes to the connecting position, the bridge portions 136 and 145 of the coupling device 82 contact the inside of the slider 84 to prevent its further slide.

The first and second receiving recesses 159 and 160 become registered with the access openings 136a and 145a of the coupling device 82, and constitute portions of the access hole 92. When the housing 81 or the flexible sheath 11 is inserted in the access hole 92, the receiving recesses 159 or 160 receive a peripheral surface of either one of the housing 81 and the flexible sheath 11, to disable the slider 84 from sliding.

A spring arm 163 is formed in an end of the lower slide plate 155, and resiliently shiftable in the transverse direction of the slider 84. A retaining hook 164 of click projects from an end of the spring arm 163 in the transverse direction of the slider 84. Retaining grooves 165 and 166 of click are formed in an edge of the through opening 133 of the lower casing 120. See FIG. 13. The retaining hook 164, when the slider 84 is in an initial position, is engaged with the retaining groove 165, and when the slider 84 is set in the connecting position, is engaged with the retaining groove 166.

A cutout 169 is formed in the upper slide plate 156 of the slider 84 to reduce its width. When the slider 84 is in the initial position, the cutout 169 is positioned between the connection opening 95 and the access opening 145a to define a path. Thus, the shaft head 57 can be moved horizontally through this path for connection with the support 48.

A shifting wall 172 is a lower wall of the upper slide plate 156, presses the shaft head 57 in the access hole 92 through the access opening 145a when slid from the initial position to the connecting position, for the support 48 to hook the shaft head 57 in the fastening clip device 18. The shifting wall 172 includes a first inclined surface 172a and a second inclined surface 172b. The first inclined surface 172a extends downwards from the cutout 169 toward the second receiving recesses 160 with reference to the insertion of the slider 84. The second inclined surface 172b extends downwards from the first inclined surface 172a toward the second receiving recesses 160 with a greater angle than the shifting wall 172.

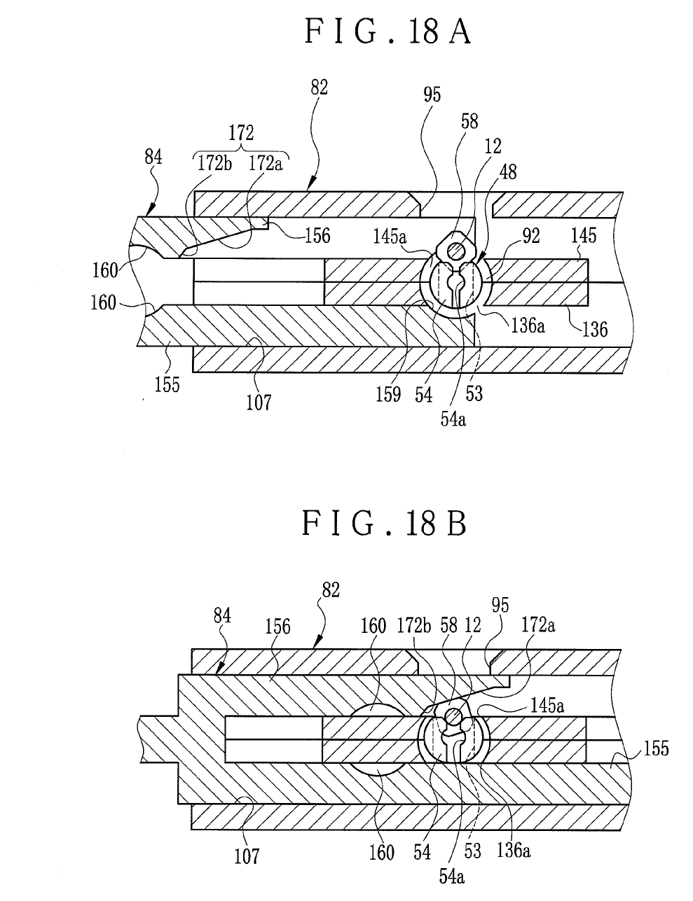

In the shaft head 57 inserted horizontally in the connection opening 95 in FIG. 18A, the lateral surface 58a of the front shaft head portion 58 is inserted through the access opening 145a into a space between the cavity walls 53 of the support 48.

In FIG. 18B, the slider 84 is pushed into the slide channel 107. The first inclined surface 172a of the shifting wall 172 contacts the shaft head portions 58 and 59 and depresses those gradually in a downward direction. The lateral surface 58a of the front shaft head portion 58 rotates in contact with the first inclined surface 172a, and becomes clamped between the cavity walls 53 deformed with resiliency. Also, the operating wire 12 becomes inserted and clamped between the clamping walls 54.

It is likely that offsetting occurs with the operating wire 12 or the support 48 to cause failure in fastening if the operating wire 12 and the shaft head 57 are pressed into the fastening mechanism 52 abruptly. However, it is possible in the invention to reduce occurrence of abnormal fastening because the shaft head 57 is entered gradually by the first inclined surface 172a having a small inclination.

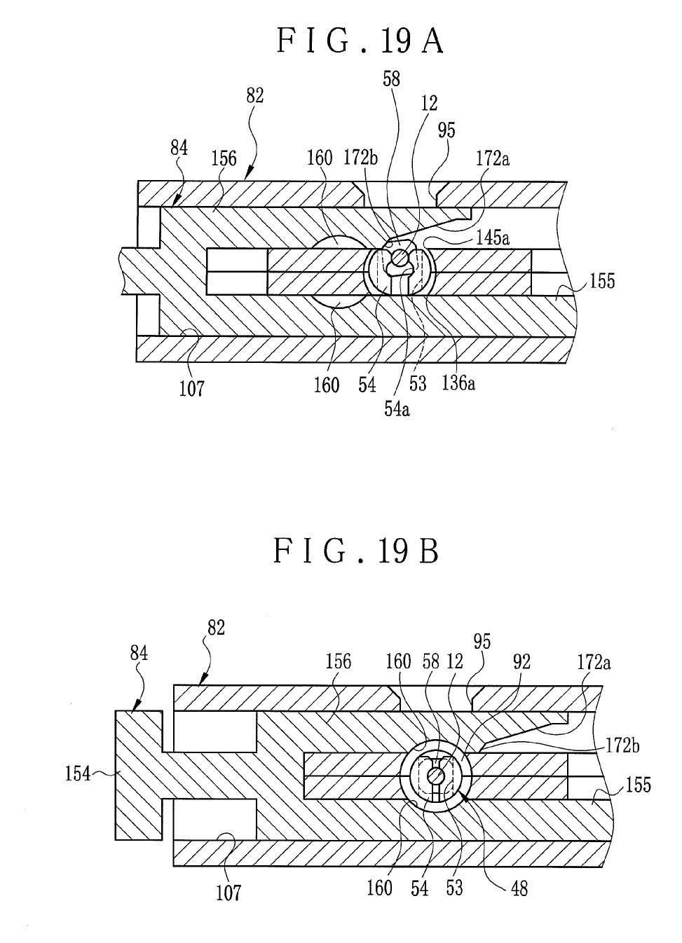

In FIG. 19A, the second inclined surface 172b pushes the shaft head portions 58 and 59 quickly downwards in response to the slide of the slider 84. In FIG. 19B, the front shaft head portion 58 becomes inserted between the cavity walls 53. The operating wire 12 becomes clamped between the clamping walls 54. The rear shaft head portion 59 comes in contact with a proximal end of the clamping walls 54.

When the housing 81 is pulled out of the coupling device 82, the fastening mechanism 52 of the support 48 is unstable within the access hole 92. However, the lower slide plate 155 of the slider 84 receives a lower portion of the fastening mechanism 52 through the access opening 136a, and can keep the fastening mechanism 52 positioned firmly even with pressure of the shifting wall 172. The upper and lower slide plates 155 and 156 of the slider 84 are retained by the coupling device 82, and can be prevented from deformation even in occurrence of reaction in the course of hooking the shaft head 57 on the support 48.

Introduction of the multiple clip assembly 13 into the housing 81 is referred to now. At first, the tubular shells 20A-20C are mounted on the clips 19A-19C to obtain the clip devices 17A-17C. The tubular shell 20 on the clips 19 is moved to a position of contact of the push sleeve 30 with the side projections 27, to protrude the turn 26 from a proximal end of the tubular shell 20. The end channels 44 are flexed outwards in the tubular shell 20, to engage the claws 23 of a second one of the clips 19 with the turn 26 of a first one of the clips 19. The tubular shell 20 is moved to set the push sleeve 30 in the initial position near to the crossed portion 24, to maintain the fastened state of the clips 19 with the tubular shell 20. Similarly, the fastening clip device 18 is connected with the clip 19C which is located on the proximal side.

The multiple clip assembly 13 is contained in the lower housing half 110 to set the fins 38 in the fin receiving slots 114. See FIG. 11. The end connector 104 of the pull rod structure 83 is also contained in the lower housing half 110. The arms 25 of the clip 19A are closed, to engage the claws 23 with the engageable projections 104c. After this, the upper housing half 111 is fitted on the lower housing half 110 to obtain the housing 81.

The coupling device 82 is constructed by assembling the upper and lower casings 120 and 121. The slider 84 is inserted in the slide channel 107, and positioned when the first receiving recesses 159 are registered with the access hole 92. Note that a point of stationary positioning of the slider 84 can be checked by the engagement of click between the retaining hook 164 and the retaining groove 165.

The pull rod structure 83 is introduced through the stage groove 91 and the access hole 92 by collapsing the pull tab portion 102. The shank 103 is inserted in the fin bending channel 99. The pull tab portion 102 comes to protrude from the coupling device 82 through the pull opening 151.

The housing 81 is inserted in the axial direction fully through the access hole 92 by advancing the exit opening 88 for inserting the key projection 117 in the key way groove 125. Thus, the barrel cavity 87 is registered internally with the fin bending channel 99 inside the coupling device 82. Note that the full insertion of the housing 81 in the access hole 92 can be checked according to the state of a click of the retaining hook 117b with the retaining opening 126 at the key way groove 125.

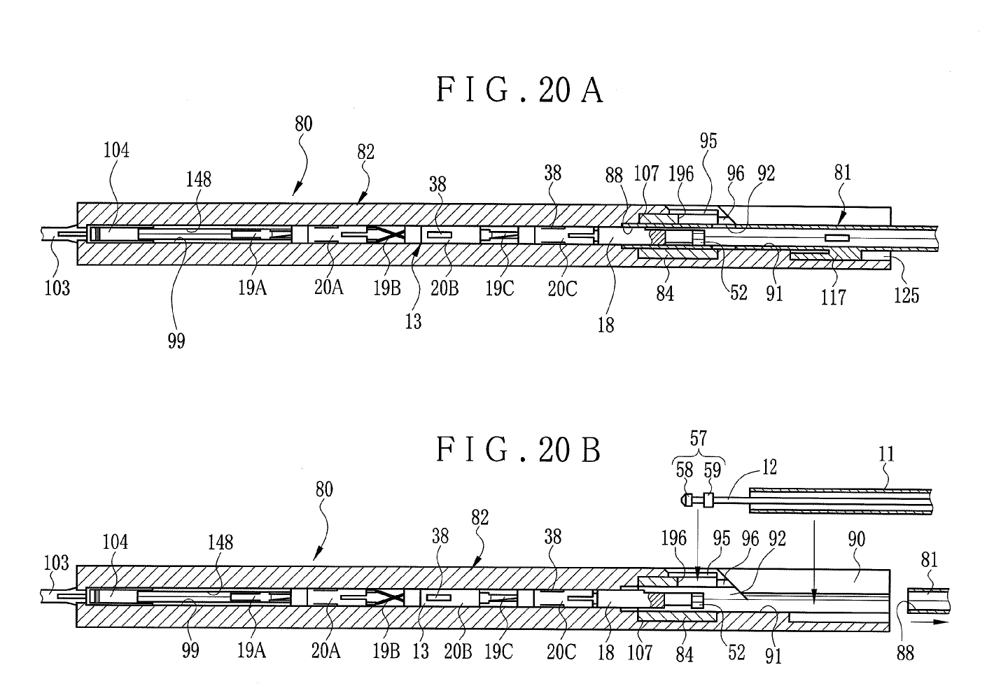

A method of loading the multiple clip assembly 13 in the flexible sheath 11 from the multiple clip package 80 is described now by referring to FIGS. 20A-24.

At first, the housing 81 and the coupling device 82 in FIG. 8 are held together by the recess 90. In FIG. 20A, the pull tab portion 102 is pulled from the coupling device 82. The end connector 104 is slid to the fin bending channel 99 by following the shank 103 pulled through the coupling device 82. The multiple clip assembly 13 is introduced in the fin bending channel 99 through the barrel cavity 87 by the pull of the end connector 104.

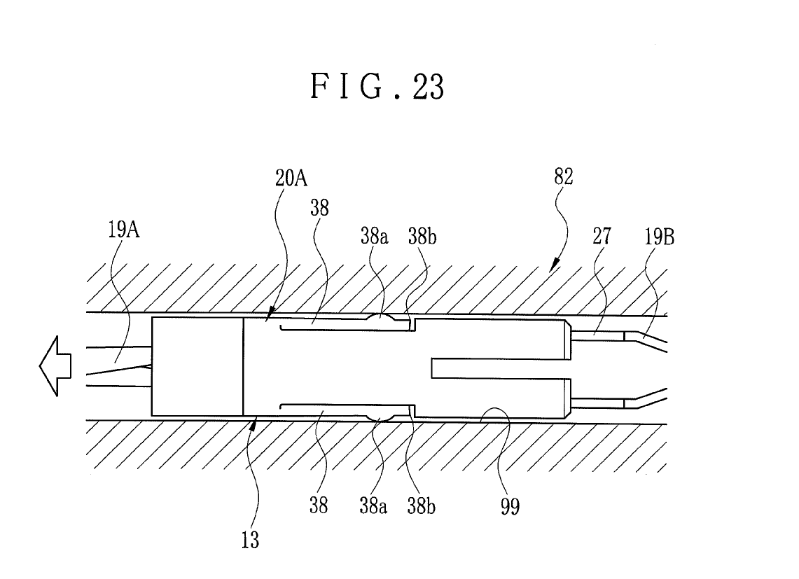

In FIG. 23, the multiple clip assembly 13 is introduced in the fin bending channel 99. The fins 38 of the tubular shell 20 are depressed and stowed by the inside of the fin bending channel 99. As the projections 38a of the fins 38 contact the inside of the fin bending channel 99, the fin ends 38b come internally lower than an outer surface of the tubular shell 20. As movement of the tubular shell 20 is limited by contact of its proximal end with the side projections 27 of the clips 19, no offsetting occurs between the clips 19 and the tubular shell 20.

In FIG. 16B, the clip 19A reaches the release groove 148. The arms 25 of the clip 19A become open to disengage the claws 23 from the end connector 104. The multiple clip assembly 13 is now stationary at a predetermined point in the fin bending channel 99. The support 48 of the fastening clip device 18 reaches the position corresponding to the connection opening 95. The pull rod structure 83 is pulled until the anti-reverse projection 103b comes to protrude from the coupling device 82 externally.

In FIG. 20B, the housing 81 is removed from the access hole 92 in the axial direction. Then a portion of the support 48 of the fastening clip device 18 appears through the access hole 92. The fastening mechanism 52 of the support 48 is opposed to the connection opening 95 through the access opening 145a and the cutout 169 in the slider 84.

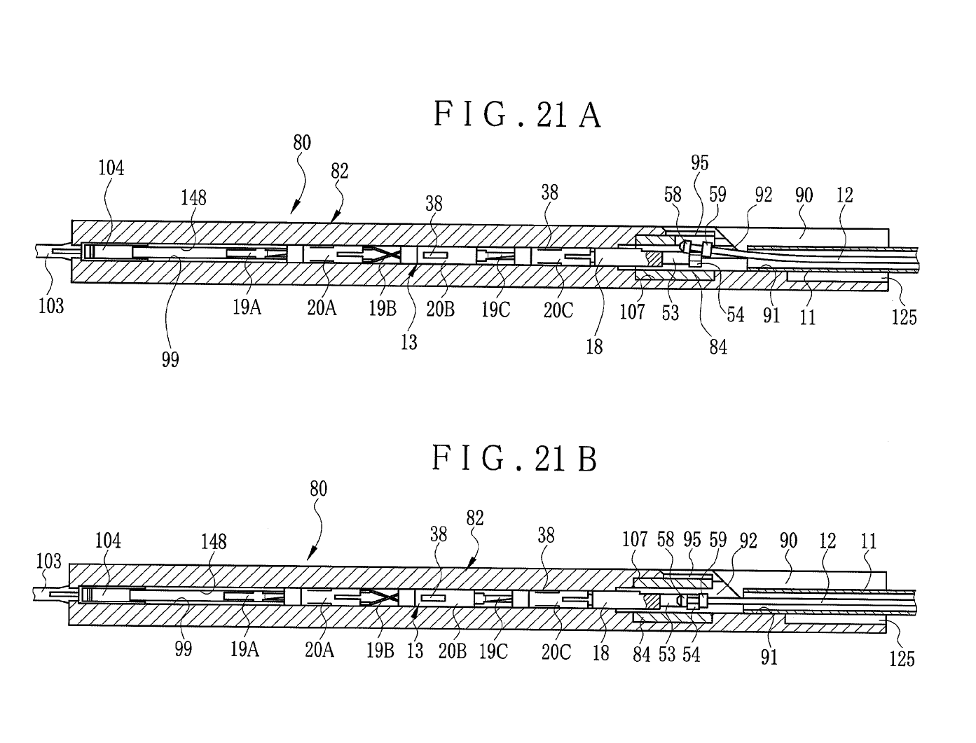

The operating wire 12 and the shaft head 57 previously protrude from the sheath end of the flexible sheath 11 according to pull of the sheath handle 63 relative to the wire handle 62. In FIGS. 18A and 21A, the flexible sheath 11, the operating wire 12 and the shaft head 57 are inserted into respectively the stage groove 91, the wire channel 96 and the connection opening 95 in the downward direction to the coupling device 82. After their insertion, the flexible sheath 11 and the coupling device 82 are retained together by use of the recess 90. Corners of the lateral surface 58a of the front shaft head portion 58 are inserted between the cavity walls 53.

In FIGS. 18B and 19A, when the slider 84 is pushed into the slide channel 107, the first and second inclined surfaces 172a and 172b of the shifting wall 172 depress the shaft head portions 58 and 59 downwards. In FIGS. 19B and 21B, the front shaft head portion 58 pushed by the slider 84 becomes inserted between the cavity walls 53. The cavity walls 53 resiliently clamp the front shaft head portion 58 for tight connection. Also, the operating wire 12 is clamped between the clamping walls 54. The rear shaft head portion 59 contacts a rear end surface of the clamping walls 54.

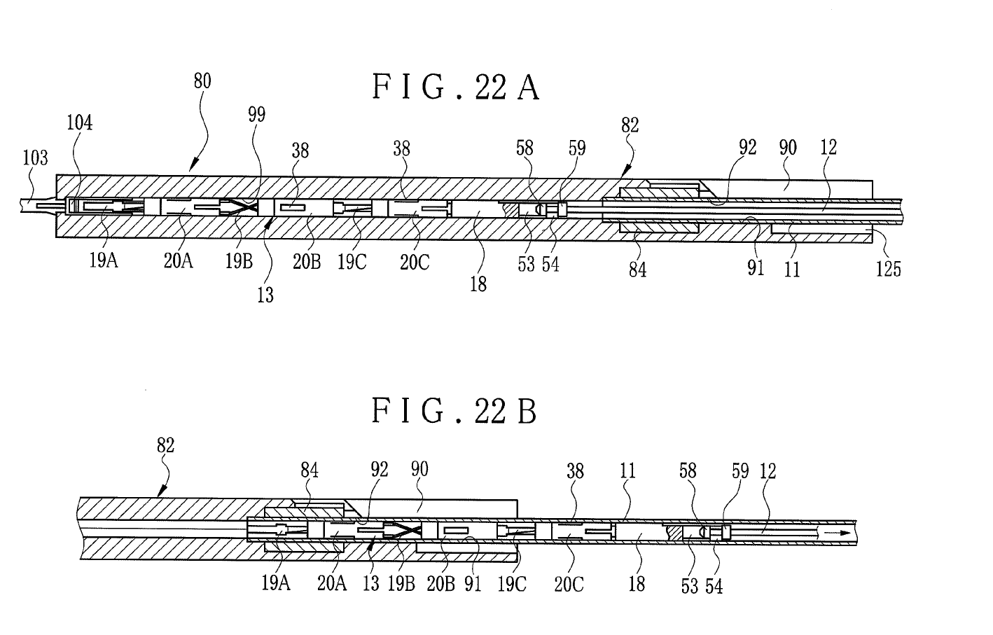

In FIG. 22A, the entirety of the multiple hemostatic clip application apparatus 10 is pushed relative to the coupling device 82. Thus, the flexible sheath 11 is pushed fully into the access hole 92, for the inner surface of the flexible sheath 11 to communicate with the inner surface of the fin bending channel 99. Also, the multiple clip assembly 13 pushed by the rear shaft head portion 59 advances through the fin bending channel 99.

A length of the fin bending channel 99 is so determined that the clip 19A after moving does not interfere with the end connector 104. Thus, the clip 19A can be free from being damaged with the fin bending channel 99. An opening direction of the clips 19 is different from a deploying direction of the fins 38 with a difference of 90 degrees with reference to an axis of the tubular shell 20. Accordingly, the fins 38 will not be opened by the release groove 148.

Then the operating wire 12 is pulled relative to the flexible sheath 11. For example, the wire handle 62 is pulled away from the sheath handle 63, so that the operating wire 12 can be moved relative to the flexible sheath 11 with a great length.

In FIG. 22B, the operating wire 12 is pulled. In response, the multiple clip assembly 13 is introduced in the flexible sheath 11 by advance of its proximal end. As the inner surface of the flexible sheath 11 is registered with the fin bending channel 99 during loading of the multiple clip assembly 13, the multiple clip assembly 13 can be moved while the fins 38 are depressed, so that the resistance can be reduced. The multiple clip assembly 13 can be loaded in the flexible sheath 11 without offsetting of the tubular shells 20A-20C from the clips 19A-19C.

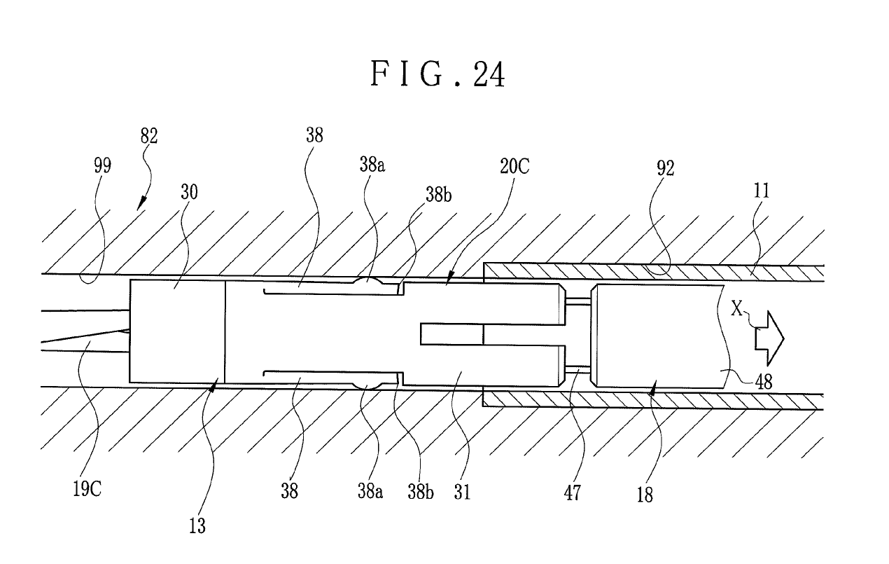

In FIG. 24, the fin ends 38b of the fins 38 are set lower than an outer surface of the tubular shell 20 by operation of the projections 38a. The fin ends 38b do not interfere with the sheath end of the flexible sheath 11 during the loading of the multiple clip assembly 13. Also, the projections 38a contact the inside of the flexible sheath 11 upon introduction of the tubular shell 20 in the flexible sheath 11. Thus, the multiple clip assembly 13 can be smoothly introduced to a predetermined position with reduced friction. There occurs no offsetting between the clips 19 and the tubular shell 20 as force of fastening of the clips 19 with the fins 38 can be sufficient by engagement of the projections 38a with the inside of the fin bending channel 99 and the flexible sheath 11.

When the sheath handle 63 is engaged with the first one of the notches 69 of the wire handle 62, loading of the multiple clip assembly 13 is completed. The flexible sheath 11 with the multiple clip assembly 13 is pulled away from the coupling device 82.

As has been described heretofore, it is possible in the multiple clip package 80 to preserve and handle the clips 19 in an assembled state, and to load the flexible sheath 11 with the clips 19 easily in the assembled state. Clip loading is possible in a short time easily without excessive load to manual handling.

The multiple clip package 80 can be preserved or handled in a state of deploying the fins 38 of the tubular shell 20. It is possible to prevent drop of resiliency of the fins 38, and to use the clips 19 and the tubular shell 20 with full performance for the purpose of tissue clamping. Also, the multiple clip assembly 13 can be loaded in the flexible sheath 11 in a state of depressing and stowing the fins 38. The tubular shell 20 can be kept with friction from moving relative to the clips 19.

The housing 81 can be prevented from rotationally shifting on the coupling device 82 by engaging the key projection 117 with the key way groove 125. The multiple clip assembly 13 can be pulled in a suitably maintained orientation. Thus, failure and degradation of the multiple clip assembly 13 due to deformation or distortion can be prevented, so as to maintain original performance of the multiple hemostatic clip application apparatus 10 with the multiple clip assembly 13. Also, the housing 81 is kept from moving in the axial direction by engaging the retaining hook 117b with the retaining opening 126. Orientation of the housing 81 can be maintained appropriately by connection with the coupling device 82.

In the above embodiment, the clip 19A of the multiple clip assembly 13 is pulled and introduced in the coupling device 82 from the housing 81. However, a proximal end of the multiple clip assembly 13 can be pushed for introduction to the coupling device 82 from the housing 81. Another preferred embodiment for loading the multiple clip assembly 13 in the coupling device 82 is described now. Elements similar to those of the above embodiments are designated with identical reference numerals.

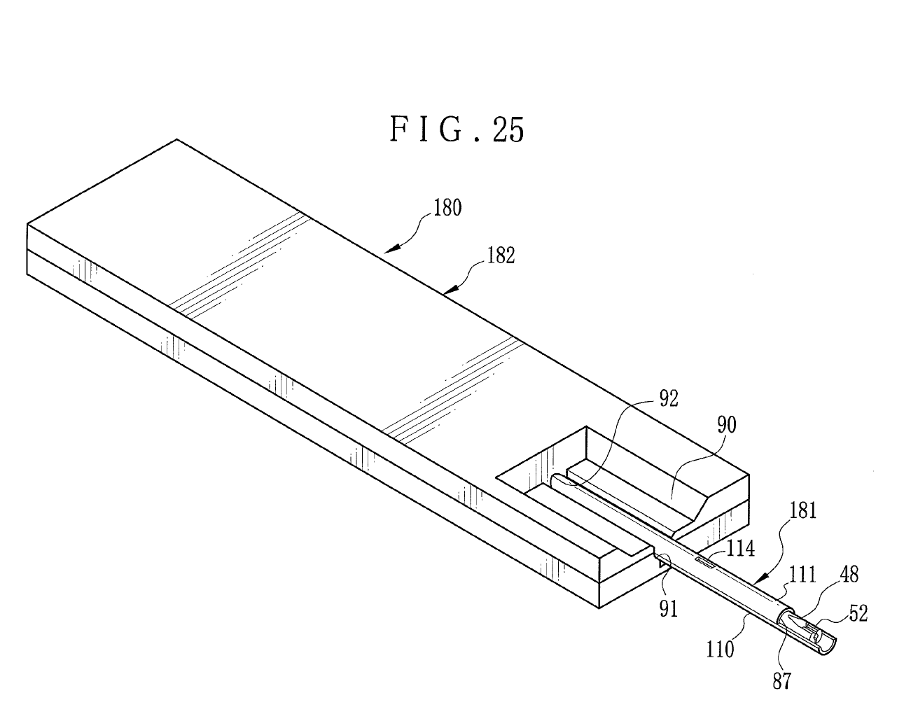

In FIG. 25, a multiple clip package 180 or multiple clip holder includes a housing 181 or barrel, and a coupling device 182. A portion of the housing 181 at the proximal end of the upper housing half 111 is shorter than the lower housing half 110. The fastening mechanism 52 as a portion of the support 48 of the fastening clip device 18 protrudes from the barrel cavity 87. The shaft head 57 in the multiple clip assembly 13 inside the housing 181 is connectable as the support 48 appears externally in the upper housing half 111. The support 48 can be protected from incidental pressure, because contained in the lower housing half 110.

The coupling device 182 does not have the connection opening 95 or the wire channel 96, as the shaft head 57 is fastened to the fastening mechanism 52 at the proximal end of the housing 181 protruding from the coupling device 182. The coupling device 182 does not have the release groove 148, the access opening 145a, the pull rod structure 83 or the slider 84 either.

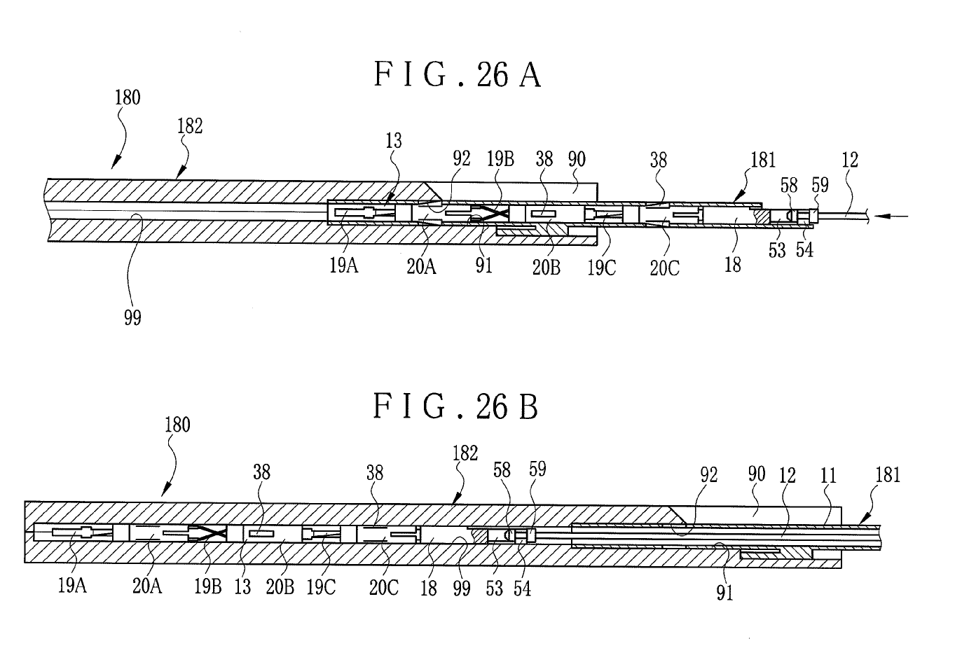

In FIG. 26A, the operating wire 12 is protruded from the flexible sheath 11. The shaft head 57 is engaged with the fastening mechanism 52. The engagement is easy because fingers can pinch the fastening mechanism 52 and the shaft head 57 for positioning.

In FIG. 26B, the operating wire 12 is inserted in the housing 181 after fastening the operating wire 12 to the multiple clip assembly 13. The multiple clip assembly 13 is pushed by the rear shaft head portion 59 to move through the housing 181, and advances into the fin bending channel 99 by moving forwards its distal end. Each of the fins 38 of the multiple clip assembly 13 is depressed and stowed by the inside of the fin bending channel 99.

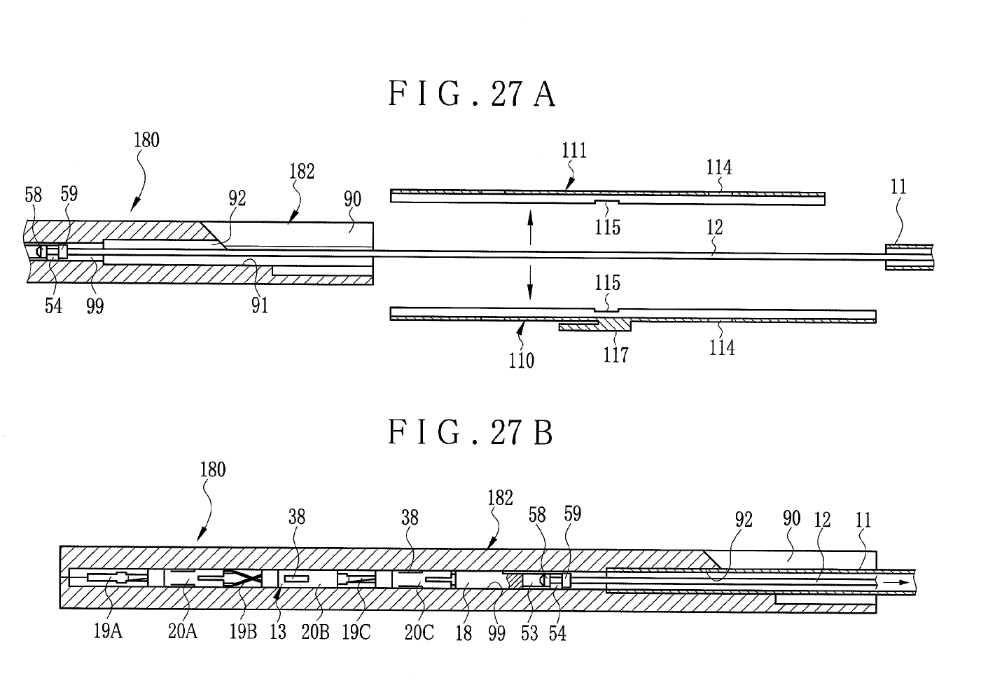

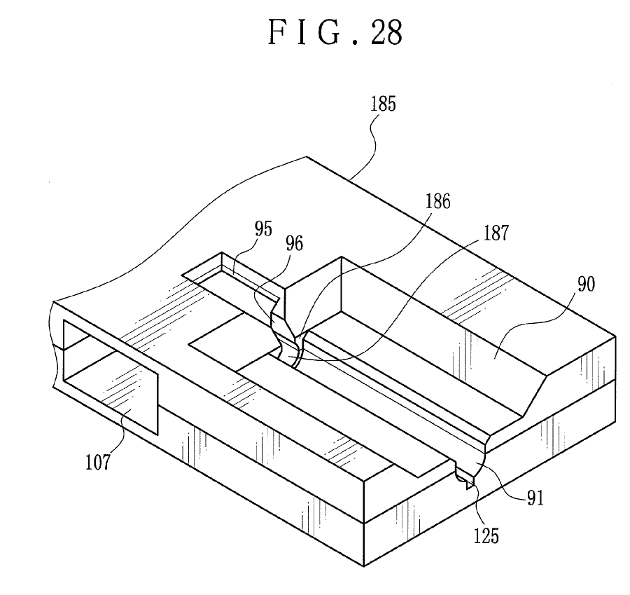

After the multiple clip assembly 13 is introduced in the coupling device 182, the housing 181 is pulled away from the access hole 92 to separate the upper housing half 111 from the lower housing half 110. See FIG. 27A. Thus, the housing 181 is removed from around the operating wire 12. It is preferable to join the lower housing half 110 with the upper housing half 111 in an easily removable manner in the housing 181.

In FIG. 27B, the flexible sheath 11 is moved toward the distal end relative to the operating wire 12, and inserted in the stage groove 91 and the access hole 92, to register the fin bending channel 99 with the inside of the flexible sheath 11. Then the operating wire 12 is pulled back relative to the flexible sheath 11. In a manner similar to the multiple clip package 80 of FIG. 22B, the multiple clip assembly 13 at the distal end of the operating wire 12 is introduced in the flexible sheath 11 and loaded therein. As a result, no offsetting will occur between the clips 19 and the tubular shell 20 in the position.

In the embodiment, the housing 81 is cylindrical as a barrel, and the coupling device 82 is plate-shaped. However, the housing 81 and the coupling device 82 may be formed in other forms. Furthermore, the shaft head 57 can be advanced in the axial direction for insertion and fastening into the fastening mechanism 52 of the fastening clip device 18 instead of downward insertion of the shaft head 57 in the above embodiment.

In addition to fastening of the clips 19 by their direct engagement, it is possible to use separate parts for fastening the clips 19 to one another in cooperation with the tubular shell 20, for example, fastening hooks or the like for clips.

In the above embodiment, the sheath end of the flexible sheath 11 is inserted in the access hole 92 for registering the fin bending channel 99 with the inside of the flexible sheath 11 before pulling the multiple clip assembly 13 from the coupling device 82 into the flexible sheath 11. However, an error in manual operation of insertion of the flexible sheath 11 in the access hole 92 may occur. For example, the sheath end is likely to advance incompletely, or to extend only to an intermediate point in the access hole 92. If the multiple clip assembly 13 enters the flexible sheath 11 in the state of the incomplete advance, it is likely that the fins 38 of the tubular shell 20 open in the course of the pull to break the clips 19 due to interference with the sheath end.

Modification of the coupling device 82 is possible in view of preventing this error in the operation by registering the inside of the flexible sheath 11 with the fin bending channel 99 while the distal end of the flexible sheath 11 is absent in the access hole 92. Still another preferred embodiment is hereinafter described in which the flexible sheath 11 does not enter the coupling device 82. Elements similar to those of the above embodiments are designated with identical reference numerals.

In FIG. 28, a coupling device 185 of the embodiment has a structure nearly the same as that of the coupling device 82. A contact surface 186 is formed at an end of the stage groove 91 for contact of a proximal end of the housing 81 or the flexible sheath 11. A fin bending channel 187 or skirt bending channel is formed in the coupling device 185, and has one end positioned at the contact surface 186. The fin bending channel 187 is defined by elongating the fin bending channel 99 of the above embodiment to the contact surface 186, and has an inner diameter equal to that of the barrel cavity 87 and the flexible sheath 11. The wire channel 96 extends to communicate with the fin bending channel 187, and has a smaller width than the fins 38 of the tubular shell 20.

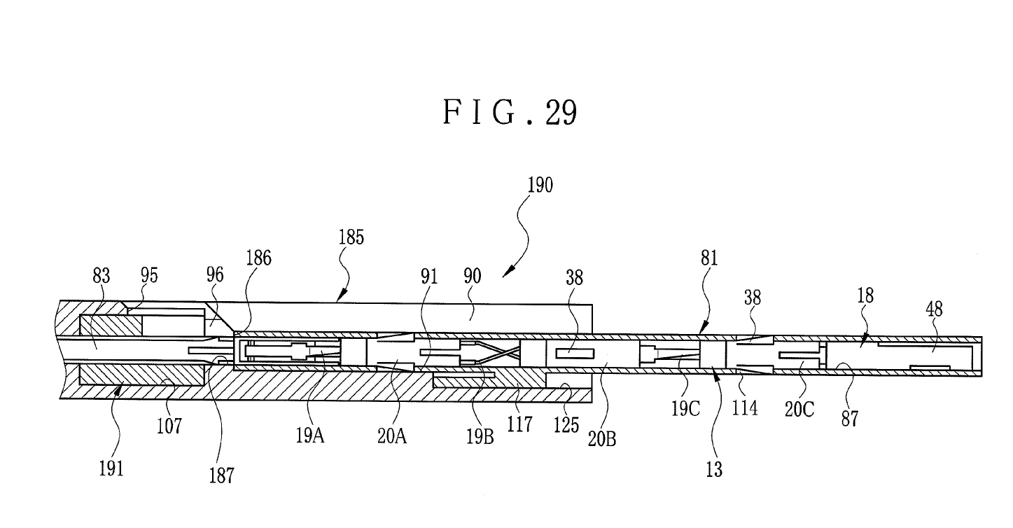

In FIG. 29, a multiple clip package 190 or multiple clip holder includes a slider 191, the housing 81, the pull rod structure 83 and the coupling device 185. In the same manner as the multiple clip package 80, the housing 81 contains the multiple clip assembly 13. The distal end of the housing 81 with the exit opening 88 contacts the contact surface 186 while the housing 81 is contained in the stage groove 91. The pull rod structure 83 is inserted in the fin bending channel 187. The pull tab portion 102 protrudes from a second end of the coupling device 185. The slider 191 has a form similar to the slider 84 of the first embodiment, and inserted in the slide channel 107.

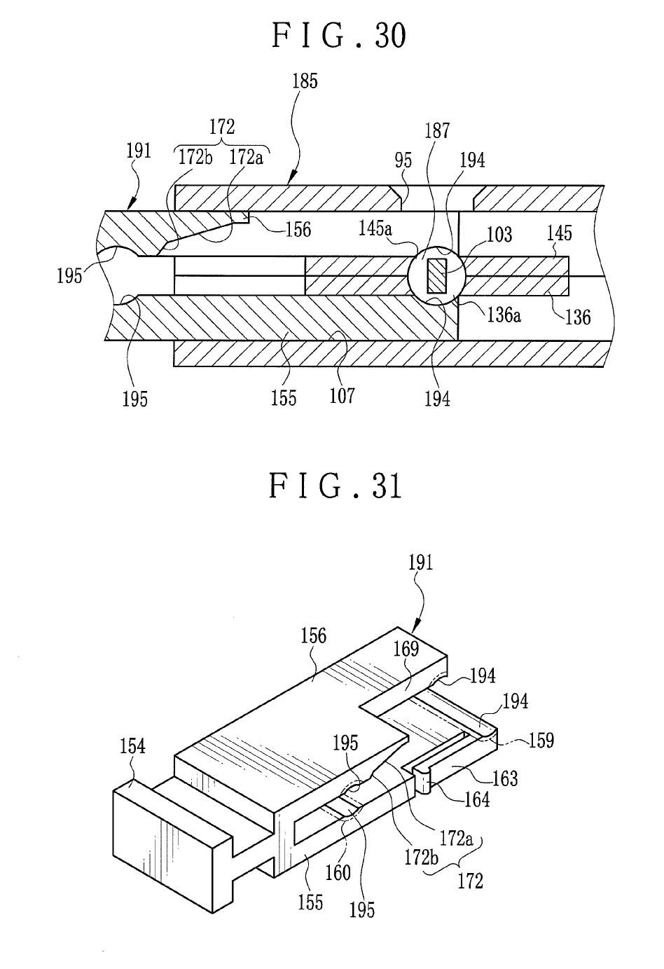

In FIG. 30, the slide channel 107 of FIG. 29 is depicted in a section. The bridge portions 136 and 145 extend through the slide channel 107 of the coupling device 185 for reinforcement similar to the coupling device 82 of the first embodiment. Portions of the fin bending channel 187 are formed in the bridge portions 136 and 145. The access openings 136a and 145a are open through upper and lower walls of the fin bending channel 187 at the bridge portions 136 and 145, to enable insertion of the shaft head 57 of the operating wire 12 inserted through the connection opening 95.

In FIG. 31, first receiving recesses 194 and second receiving recesses 195 are formed in the slider 191, and have a diameter equal to that of the fin bending channel 187, in the same manner as the slider 84 with the first and second receiving recesses 159 and 160. In FIG. 30, the first receiving recesses 194 define a portion of the fin bending channel 187, and are registered with the access openings 136a and 145a while the slider 191 is slid externally from the slide channel 107 before use of the multiple clip package 190.

In a manner similar to the multiple clip package 80 described above, the multiple clip assembly 13 is introduced from the housing 81 into the coupling device 185 as the pull rod structure 83 is pulled relative to the coupling device 185. As the barrel cavity 87 in the housing 81 is registered with the fin bending channel 187, the fins 38 of the tubular shell 20 are kept stowed and depressed during the introduction in the fin bending channel 187.

During introduction of the multiple clip assembly 13 into the coupling device 185, one of the fins 38 directed upwards is opposed to the wire channel 96. However, the fins 38 do not become deployed because the wire channel 96 has a width smaller than that of the fins 38.

In the fin bending channel 187 of the slide channel 107, the cutout 169 of the slider 191 makes the access opening 145a accessible for the purpose of insertion of the shaft head 57. The fins 38 directed upwards are likely to deploy temporarily in the access opening 145a. However, the tubular shells 20 are prevented from moving backwards by the side projections 27 of the clips 19 disposed on the proximal side. Even when the fins 38 are deployed temporarily during advance of the multiple clip assembly 13 toward the distal end, the multiple clip assembly 13 is maintained in the fastened state.

After the multiple clip assembly 13 is moved, the housing 81 is removed from the coupling device 185. In the multiple clip package 190 of the embodiment, the exit opening 88 of the housing 81 is not inserted deeply in the coupling device 185 in the manner of the multiple clip package 80 of the first embodiment. It is possible to remove the housing 81 from the coupling device 185 easily by moving upwards.

The flexible sheath 11 is inserted in the stage groove 91 in the coupling device 185 upon introduction of the multiple clip assembly 13. The sheath end is pressed on the contact surface 186. The shaft head 57 and the operating wire 12 are inserted in respectively the connection opening 95 and the wire channel 96. As the contact surface 186 for contact of the sheath end can be seen by an operator, it is possible to reduce errors in manual handling in comparison with the first embodiment of insertion into the access hole 92.

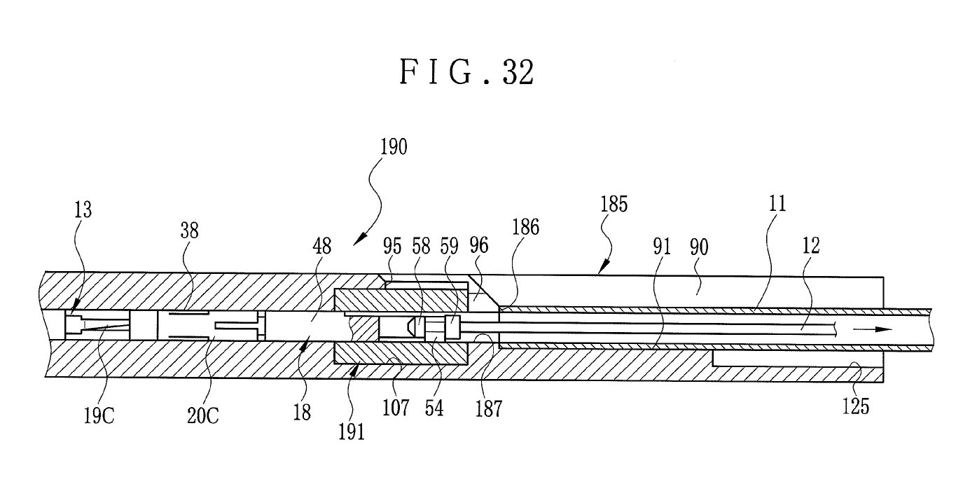

The slider 191, when pressed into the slide channel 107, presses the shaft head 57 in a manner similar to the multiple clip package 80. The shaft head 57 becomes engaged with the support 48 to fasten the multiple clip assembly 13 to the operating wire 12 as illustrated in FIG. 32.

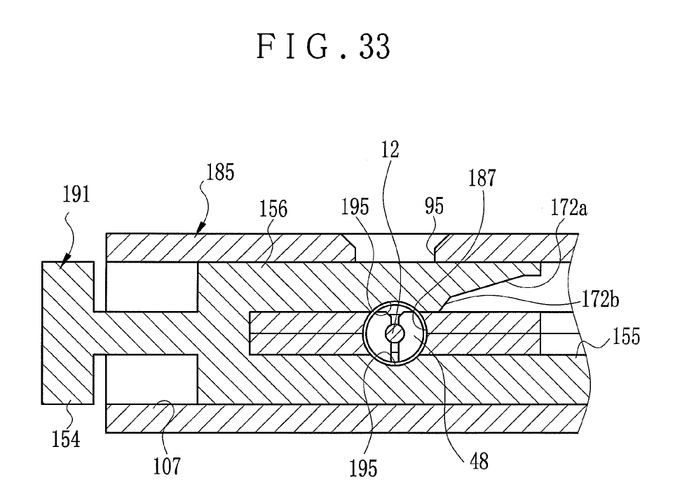

In FIG. 33, the slide channel 107 after inward slide of the slider 191 is illustrated in a section. The second receiving recesses 195 are registered with the access openings 136a and 145a and come to constitute a portion of the fin bending channel 187. A fully closed surface of the fin bending channel 187 is formed with reference to its circumference.

When the operating wire 12 is pulled in the same manner as the multiple clip package 80 of the first embodiment, the multiple clip assembly 13 is introduced in the flexible sheath 11 through the fin bending channel 187. The fin bending channel 187 is registered with the inside of the flexible sheath 11 by pressing the sheath end of the flexible sheath 11 on the contact surface 186. The multiple clip assembly 13 can be moved while the fins 38 remain depressed and stowed. The multiple clip assembly 13 can be loaded in the flexible sheath 11 without offsetting between the clips 19A-19C and the tubular shells 20A-20C.

In the first embodiment, it is likely that the multiple clip assembly 13 is erroneously positioned with a deviation of the support 48 from a fastening position upon pull of the pull rod structure 83, as an error may occur in disengaging the clip 19A from the end connector 104 within the release groove 148. Specifically, shock or vibration in the course of containment in the housing 81 may have changed a fastened state between the clip 19A and the end connector 104 as a background of the failure of the disengagement. Should the speed of the pull be very high in comparison with an expected speed level, the clip 19A may not open in an expected time sequence, because the pull rod structure 83 is pulled manually by an operator.

Also, it is likely that the multiple clip assembly 13 is broken by pressing the pull rod structure 83 into the coupling device 82. It is impossible to push the pull rod structure 83 into the coupling device 82 when the pull rod structure 83 is in an initial position before being pulled from the coupling device 82. Should the pull rod structure 83 or the coupling device 82 be pushed in the state of only small incidental protrusion of the pull rod structure 83 from the coupling device 82, the pull rod structure 83 is likely to break the clip 19A by pushing strongly. Also, the multiple clip assembly 13 may be broken by the pull rod structure 83 when the pull rod structure 83 is pushed incidentally into the coupling device 82 after introduction of the multiple clip assembly 13 in the coupling device 82.

To solve such a problem, it is preferable to retain the pull rod structure 83 in a predetermined position. Still another preferred embodiment is hereinafter described. Elements similar to those of the above embodiment are designated with identical reference numerals.

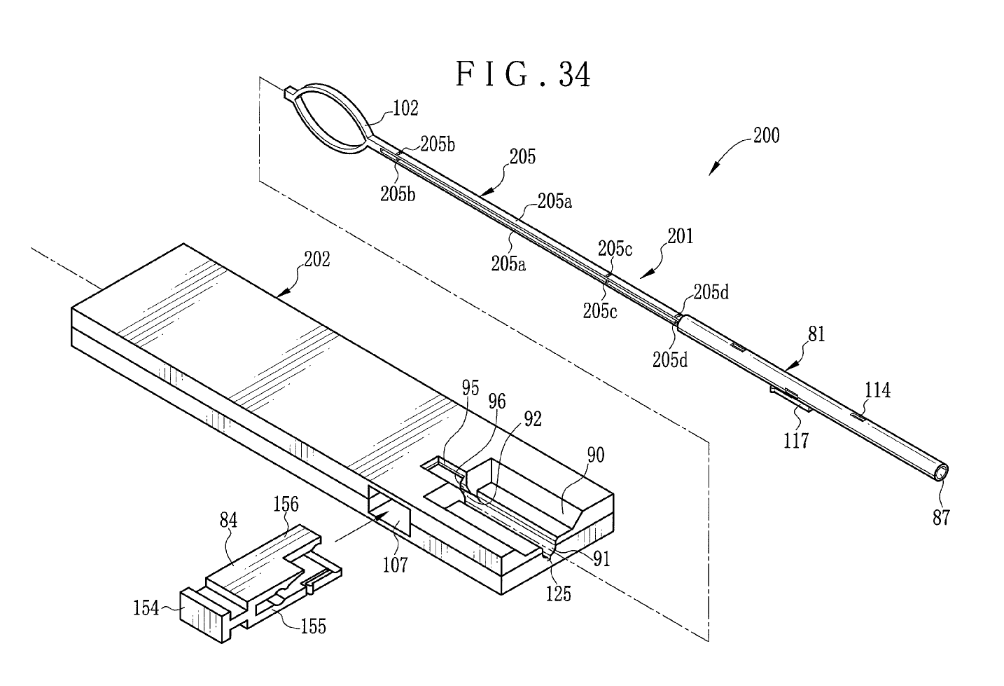

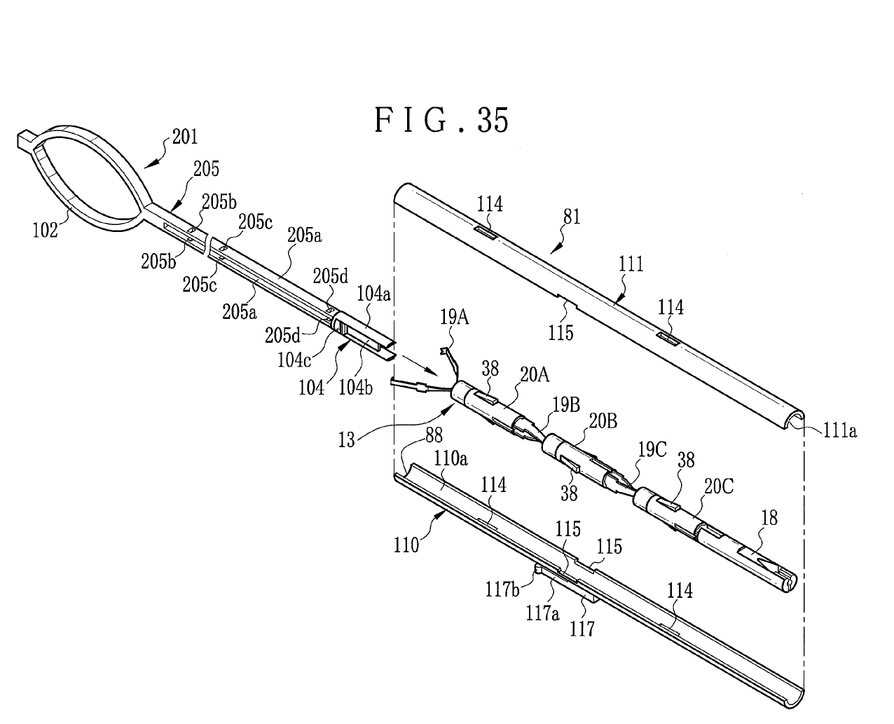

In FIGS. 34 and 35, a multiple clip package 200 or multiple clip holder has the multiple clip assembly 13, the housing 81 and the slider 84 the same as those of the first embodiment, and also includes a pull rod structure 201 and a coupling device 202.

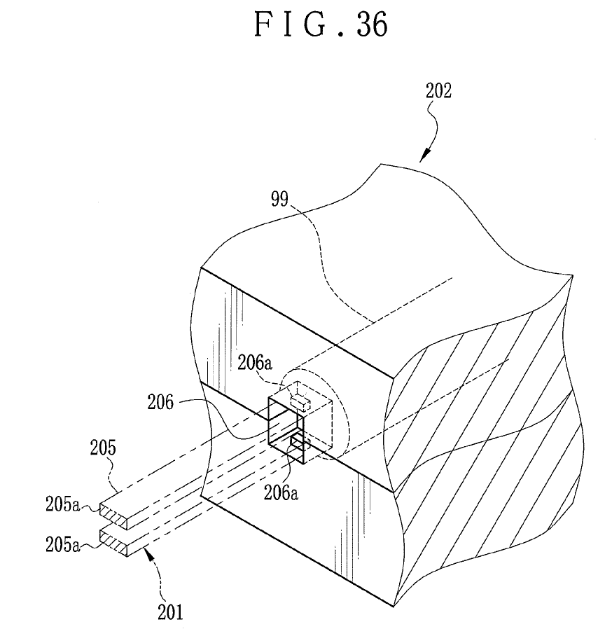

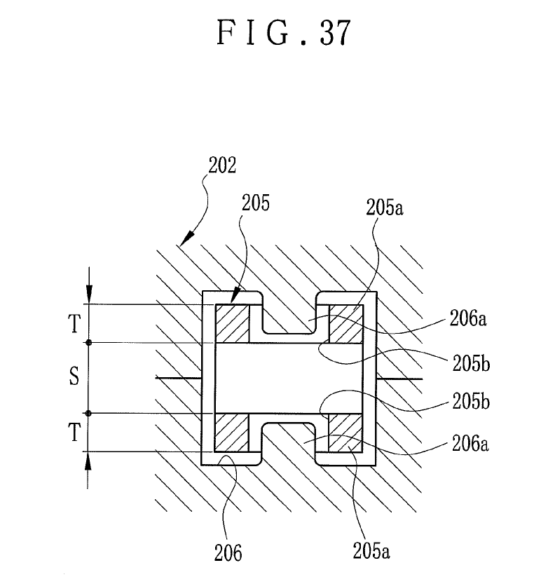

A shank 205 is a portion of the pull rod structure 201. A pair of elongated plates 205a constitute the shank 205, are resilient, extend in the axial direction of the fin bending channel 99 in the coupling device 202, and are disposed with a small space at the center as viewed in a transverse direction. In FIGS. 36 and 37, a pull opening 206 is formed in the coupling device 202, and has a form similar to a shape defined by outer edges of the elongated plates 205a as viewed in a cross section inclusive of the small space between those. Thus, the shank 205 is engaged with the pull opening 206 in an anti-rotation state about an axis of the axial direction.

It is likely that the pull tab portion 102 twists relative to the coupling device 202 in the course of pulling the pull tab portion 102 from the coupling device 202. However, the shank 205 does not rotate in the pull opening 206, so as to block transmission of the twist of the pull tab portion 102 to the end connector 104. Therefore, it is possible to prevent failure and damage of the multiple clip assembly 13, for example, distortion of the arms 25 of the clip 19A, disengagement of the clips 19 or the like. The shank 205 absorbs the twist of the pull tab portion 102 relative to the coupling device 202 between the pull tab portion 102 and the pull opening 206 by deformation of the elongated plates 205a with a shift in a space between those. Thus, working efficiency can be high as the pull tab portion 102 can be pulled and twisted simultaneously.

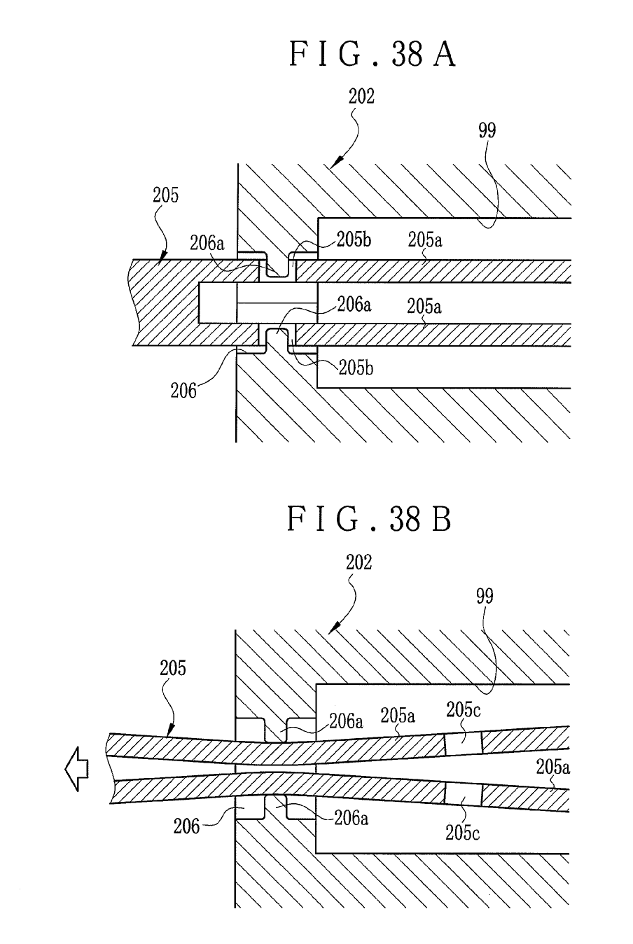

In FIG. 35, pairs of retaining holes 205b, 205c and 205d are formed in the elongated plates 205a and positioned equally in the axial direction. In FIGS. 37 and 38A, projections 206a are formed on upper and lower surfaces of the pull opening 206. The retaining holes 205b of the pull tab portion 102 are engaged with the projections 206a when the pull rod structure 201 is in an initial position without pull from the coupling device 202. This prevents incidental pull of the pull rod structure 201 from the coupling device 202 before use of the multiple clip package 200.

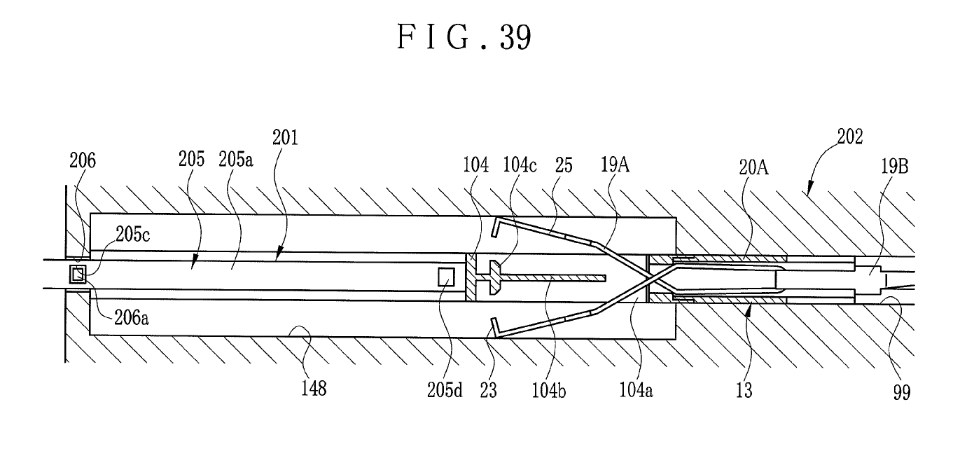

The retaining holes 205c disposed secondly on the side of the pull tab portion 102 are engaged with the projections 206a when the pull rod structure 201 is pulled to a release position where the clip 19A reaches the release groove 148 in FIG. 39. The pull rod structure 201 is temporarily stopped in the release position. The end connector 104 can be reliably disengaged from the clip 19A irrespective of pulling speed of the pull rod structure 201 or the fastened state between the clip 19A and the end connector 104. Precision in positioning the multiple clip assembly 13 in the coupling device 82 can be high.

The retaining holes 205d near to the end connector 104 become engaged with the projections 206a when the end connector 104 is pulled to an end position of contacting an end point of the fin bending channel 99. Thus, it is possible to prevent damage of the multiple clip assembly 13 due to incidental return of the pull rod structure 201 into the coupling device 202 after the multiple clip assembly 13 is introduced to the coupling device 202.

Only the elongated plates 205a are deformed easily to disengage the retaining holes 205b-205d in the shank 205 from the projections 206a. Thus, the working efficiency can be high.

In FIG. 38B, the elongated plates 205a are flexed by contact with the projections 206a while the pull rod structure 201 is pulled. It is likely that the elongated plates 205a contact one another in case of combination of flexing of the elongated plates 205a and twist of the pull tab portion 102. A thickness T and an interval S of the elongated plates 205a of FIG. 37 are preferably determined for engagement with the pull opening 206 in the rotational direction and for sufficiency in its strength and resiliency even in deformation of the elongated plates 205a to contact one another.

As has been described heretofore, the pull rod structure 201 in the multiple clip package 200 of the invention is retained by engagement of the projections 206a with the retaining holes 205c upon reach of the clip 19A to the release groove 148. This is effective in ensuring disengagement of the clip 19A from the end connector 104 irrespective of the fastened state between the clip 19A and the end connector 104 or a speed of pulling the pull rod structure 201. Precision in stationary positioning of the multiple clip assembly 13 in the coupling device 202 can be high.

The pull rod structure 201 is prevented from pulling incidentally from the coupling device 202, because the retaining holes 205b are engaged with the projections 206a in the initial position. The pull rod structure 201, when pulled to the end position of the fin bending channel 99, can be retained by engaging the retaining holes 205d with the projections 206a. This is effective in preventing damage of the multiple clip assembly 13 by the pull rod structure 201 accidentally pushed in the coupling device 202.

It is possible to disengage the projections 206a from the retaining holes 205b-205d of the shank 205 only by deforming the elongated plates 205a. The working efficiency can be high owing to the easy disengagement.

In the above embodiment, the retaining holes 205b-205d are formed in the shank 205. The projections 206a are formed inside the pull opening 206. Alternatively, a projection may be formed with the shank 205, and a retaining hole may be formed in an inner surface of the pull opening 206. In the above embodiment, the retaining holes 205b are formed for engaging with the projections 206a when the pull rod structure 201 is in the initial position. However, it is possible to retain the pull rod structure 201 in the initial position by use of the slider 84 or the like, and not to form the retaining holes 205b in combination with this structure.

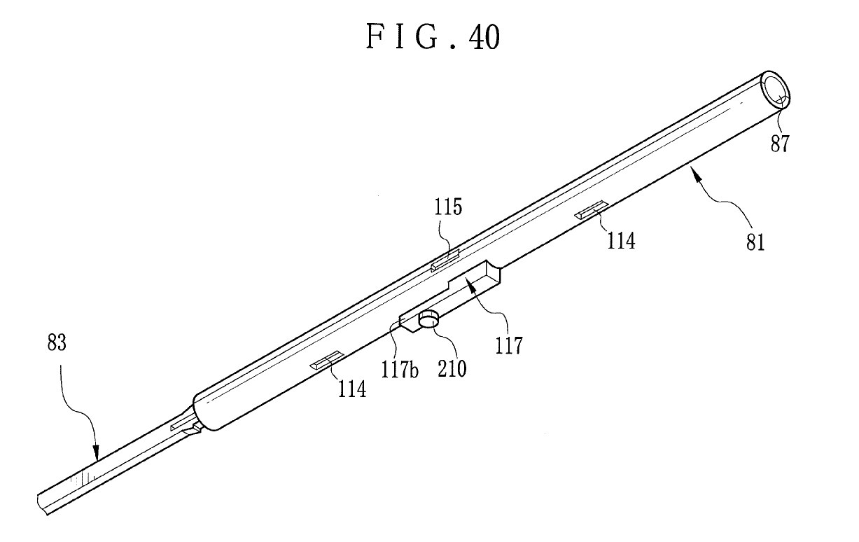

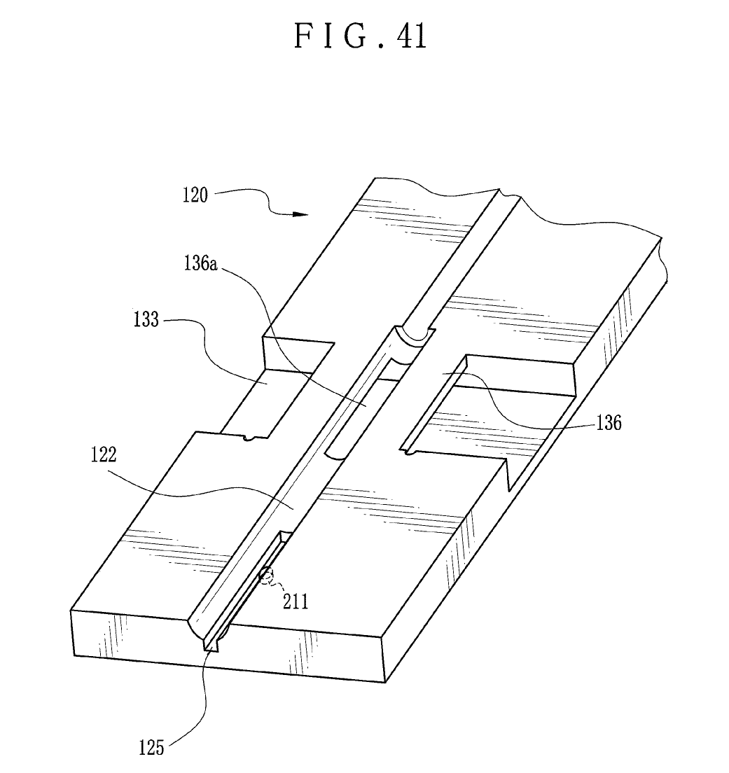

Also, it is possible to prevent the housing 81 from moving in the axial direction in a structure different from the first embodiment, in which the retaining hook 117b is engaged with the retaining opening 126 at the key way groove 125. In FIGS. 40 and 41, another preferred embodiment is illustrated. A retaining projection 210 of click is formed on a lower surface of the key projection 117. A retaining hole 211 is formed in a lower surface of the key way groove 125, and receives insertion of the retaining projection 210, to prevent the housing 81 from moving in the axial direction.

The invention is not limited to the above embodiments of the multiple clip package and clip coupling method. Various alterations and modifications are possible in the scope of the invention. Furthermore, an endoscope for use with the multiple clip application apparatus of the invention may be a rigid endoscope instead of a flexible endoscope, the apparatus being any one of a multiple clip application apparatus including a multiple clip package of the invention, and a multiple clip application apparatus operable according to a clip coupling method of the invention.

In the above embodiments, the flexible sheath has an inner bore determined regularly. However, a clip coupling method of the invention may be for use with a flexible sheath in which its inner bore is greater than a bore of its end opening for bending the fins to close and than an outer diameter of the fins in an open state.

The following are embodiment modes according to the preferred embodiments disclosed heretofore.

1. A clip coupling method for a multiple clip assembly including a plurality of clips arranged in one train and fastened to one another, comprising steps of:

introducing the multiple clip assembly from a housing containing the multiple clip assembly into a coupling device retained on the housing;

fastening a distal end of an operating wire to a proximal end of the multiple clip assembly, the operating wire being inserted through a flexible sheath movably back and forth;

pulling the operating wire relative to the flexible sheath to move a proximal end of the multiple clip assembly forwards for introduction into the flexible sheath.

2. A clip coupling method as defined in embodiment mode 1, wherein the multiple clip assembly includes plural tubular shells loadable in the flexible sheath together with the clips;

the tubular shells have a fin portion for deploying outwards with resiliency upon moving out of a sheath end of the flexible sheath with the clips, to engage with the distal end of the flexible sheath;

one of the tubular shells shifts and closes one of the clips when the clips move backwards toward an inside of the flexible sheath.

3. A clip coupling method as defined in embodiment mode 2, wherein the coupling device includes a fin bending channel, having an inner diameter substantially equal to an inner diameter of an end opening of the flexible sheath, for containing the multiple clip assembly in the step of introducing the multiple clip assembly from the housing, and for depressing and stowing the fin portion inwards.

4. A clip coupling method as defined in embodiment mode 3, further comprising a step of removing the housing from the coupling device before the step of fastening the operating wire to the proximal end of the multiple clip assembly.

5. A clip coupling method as defined in embodiment mode 3 or 4, further comprising a step of, after the step of fastening the operating wire to the proximal end of the multiple clip assembly, retaining a sheath end of the flexible sheath to the coupling device for positioning the fin bending channel in registration with an opening in the sheath end.

6. A clip coupling method as defined in any one of embodiment modes 3-5, wherein when the operating wire is pulled, the multiple clip assembly is introduced into the flexible sheath by advancing the proximal end thereof in a state of depressing and stowing the fin portion.

7. A multiple clip package comprising:

a multiple clip assembly including plural clips arranged in one train and fastened to one another;

a housing for containing the multiple clip assembly;

a coupling device for connection with the housing, and for receiving insertion of the multiple clip assembly moved by advance of a distal end thereof.

8. A multiple clip package as defined in embodiment mode 7, wherein the multiple clip assembly includes plural tubular shells loaded in a flexible sheath together with the clips;

the tubular shells have a fin portion for deploying outwards with resiliency upon moving out of a sheath end of the flexible sheath with the clips, to engage with the sheath end of the flexible sheath;

one of the tubular shells shifts and closes one of the clips when the clips move toward an inside of the flexible sheath.

9. A multiple clip package as defined in embodiment mode 8, wherein the housing includes:

a barrel cavity, having an inner diameter substantially equal to an inner diameter of an end opening of the flexible sheath, for containing the multiple clip assembly;

a fin receiving slot or recess, formed inside the barrel cavity, for receiving the fin portion of the multiple clip assembly in a deployed state in the barrel cavity; and

an exit opening for advancing the multiple clip assembly outwards from the barrel cavity.

10. A multiple clip package as defined in embodiment mode 9, wherein the coupling device includes:

a stage portion for connection with the housing; and

a fin bending channel, having an inner diameter substantially equal to an inner diameter of the barrel cavity, positioned in registration with the barrel cavity at the exit opening upon connection of the housing with the stage portion, for depressing and stowing the fin portion inwards in the multiple clip assembly introduced from the barrel cavity.

11. A tubular shell for mounting in a flexible sheath of an apparatus together with a clip including openable claws at a clip end, comprising:

a fin portion for stowing inside the flexible sheath, and for deploying further than an inner diameter of the flexible sheath after passage of a sheath end of the flexible sheath to prevent backward movement thereof;

a projection, formed on the fin portion, for contacting an inner surface of the flexible sheath upon stowing the fin portion; and

a push sleeve for pushing the clip end of the clip inserted from a proximal end of the flexible sheath when the fin portion is open after passage of the sheath end of the flexible sheath, for closing the claws.

12. A tubular shell as defined in embodiment mode 11, further comprising a portion for covering a fastened portion between a plurality of the clip fastened in one train, to maintain a fastened state of the clip.

13. A tubular shell as defined in embodiment mode 11 or 12, wherein the push sleeve is mounted around the clip, and the fin portion pushes and retains the clip when in a stowed position.

14. A tubular shell as defined in any one of embodiment modes 11-13, wherein the projection projects in an arcuate shape with reference to an axial direction in the flexible sheath.

15. A tubular shell as defined in any one of embodiment modes 11-14, wherein the fin portion is constituted by a plurality of fin portions arranged in a circumferential direction.

16. A multiple clip application apparatus comprising:

a flexible sheath of a tubular shape;

plural clips arranged in one train and fastened to one another;

a plurality of the tubular shell as defined in any one of embodiment modes 11-15, and contained in the flexible sheath with the clips;

an operating wire, fastened to a rearmost one of the clips in the flexible sheath, for pulling the plural clips.

In the multiple clip assembly, a problem is likely to occur in the fastened state of the clips because of offsetting between the clips with shock or vibration during containment in the housing, or because of distortion of the clips in the housing. Serious influence will occur in operation of the tissue clamping if the multiple clip assembly with such a problem in the fastened state is coupled with a flexible sheath.

According to the clip coupling method and multiple clip package according to embodiment mode 1 or 7, the multiple clip assembly is loaded in the flexible sheath by use of the coupling device. A structure for regulating the fastened state of clips may be added to the coupling device, and can effectively prevent occurrence of failure in the course of tissue clamping. Examples of such regulating structures include a groove, projection or the like, disposed in a space containing the multiple clip assembly within the coupling device, for limiting an interval between the clips, their rotational positions, and the like.

Also, the features of embodiment mode 1 or 7 may be constructed in combination with a multiple clip assembly of JP-A 2008-049198 without a tubular shell for pushing a clip, and clip coupling of JP-A 2006-187391 to a flexible sheath by use of a tubular shell without fin portions.

It is possible to ensure the fastened state between clips by use of the tubular shell as set forth in any one of embodiment modes 11-15. The clips can be prevented from incidental disengagement specifically in the course of passage of the flexible sheath in a tortuous portion of the endoscope with a small radius of curvature.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A Clip coupling method of loading a multiple clip assembly in a flexible sheath of a tubular shape, wherein said multiple clip assembly includes a plurality of clips arranged in one train and fastened to one another, and a plurality of tubular shells loaded in said flexible sheath together with respectively said clips, and said tubular shells have a fin portion for deploying with resiliency upon advance through a sheath end of said flexible sheath with one of said clips, and for engaging with said sheath end, one of said tubular shells shifts and closes one of said clips when said clips move back toward an inside of said flexible sheath, said clip coupling method comprising steps of:
introducing said multiple clip assembly from a housing into a fin bending channel by advancing a distal end thereof, said housing containing said multiple clip assembly in a state of deploying said fin portion, said fin bending channel having an inner diameter substantially equal to an inner diameter of an end opening of said sheath end of said flexible sheath in connection of a coupling device with said housing, said fin bending channel depressing and stowing said fin portion;
removing said housing from said coupling device, to fasten a distal end of an operating wire inserted movably through said flexible sheath to a proximal end of said multiple clip assembly;
positioning said end opening of said flexible sheath in registration with said fin bending channel; and
pulling said operating wire relative to said flexible sheath, to introduce said multiple clip assembly into said housing by advancing said proximal end thereof in a state of depressing said fin portion.

2. A clip coupling method as defined in claim 1, wherein said multiple clip assembly is introduced from said housing into said coupling device by pull of a first one of said clips.

3. A clip coupling method as defined in claim 1, wherein said multiple clip assembly is introduced from said housing into said coupling device by push of a rear one of said clips.

4. A clip coupling method as defined in claim 1, wherein said tubular shells are disposed around respectively said clips, partially cover a rear one of said clips for maintaining a fastened state thereof, and are prevented from moving backwards by said rear clip.

5. A clip coupling method as defined in claim 4, wherein said fin portion pushes and retains one of said clips in said tubular shells when depressed and stowed.

6. A multiple clip package including a multiple clip assembly, wherein said multiple clip assembly has a plurality of clips arranged in one train and fastened to one another, and a plurality of tubular shells loaded in a flexible sheath of a tubular shape together with respectively said clips, and said tubular shells have a fin portion for deploying with resiliency upon advance through a sheath end of said flexible sheath with one of said clips, and for engaging with said sheath end, one of said tubular shells shifts and closes one of said clips when said clips move back toward an inside of said flexible sheath, said fin portion is deployed while said multiple clip assembly is contained, and is depressed and stowed by loading of said multiple clip assembly in said flexible sheath, said multiple clip package comprising:
a housing, having a barrel cavity for containing said multiple clip assembly, said barrel cavity having an inner diameter substantially equal to an inner diameter of an end opening of said sheath end of said flexible sheath, and including a fin receiving opening or recess and an exit opening, said fin receiving opening or recess containing said fin portion of said multiple clip assembly in a deployed state, said exit opening causing said multiple clip assembly to move to an outside of said barrel cavity by advancing a distal end of said multiple clip assembly; and
a coupling device, having a stage portion and a fin bending channel, said stage portion receiving said housing mounted thereon, said fin bending channel having an inner diameter substantially equal to an inner diameter of said barrel cavity, wherein said fin bending channel is positioned in registration with said exit opening of said barrel cavity upon mounting said housing on said stage portion, and depresses and stows said fin portion upon introduction of said multiple clip assembly from said barrel cavity.

7. A multiple clip package as defined in claim 6, wherein said coupling device includes a clip moving structure for introducing said multiple clip assembly from said barrel cavity into said fin bending channel by advancing a distal end thereof.

8. A multiple clip package as defined in claim 7, wherein said clip moving structure includes:
a pull tab portion pullable relative to said coupling device;
a shank, having said pull tab portion positioned at a first end thereof, and inserted in said fin bending channel through a pull opening formed in said coupling device;
an end connector, disposed at a second end of said shank, for engaging with a first one of said clips in said multiple clip assembly, and for drawing said first clip when said pull tab portion is pulled, to introduce said multiple clip assembly from said barrel cavity into said fin bending channel.

9. A multiple clip package as defined in claim 8, wherein a first one of said clips in said multiple clip assembly is engaged with said end connector by keeping claws closed with pressure of an inner surface of said barrel cavity, said claws being open when in a free state without receiving external force;
said fin bending channel has a release groove for shifting said first clip to open with resiliency of said first clip when said multiple clip assembly is introduced and set in a predetermined position in said fin bending channel, to disengage said first clip from said end connector.

10. A multiple clip package as defined in claim 9, wherein an opening direction of said first clip is different from a deploying direction of said fin portion of said tubular shell associated with said first clip and with a difference of substantially a 1/4 rotation with respect to an axial direction of said tubular shell;
said release groove extends in said opening direction of said first clip.

11. A multiple clip package as defined in claim 10, wherein said multiple clip assembly includes:
a fastening clip for engaging with a rear one of said clips;
a fastening mechanism for supporting said fastening clip and for fastening to an operating wire inserted in said flexible sheath.

12. A multiple clip package as defined in claim 11, wherein said coupling device includes a wire channel and a connection opening for receiving insertion of said operating wire and said shaft head transversely to said axial direction of introducing said multiple clip assembly into said fin bending channel;
said fastening mechanism is positioned at said connection opening when said multiple clip assembly is introduced in said fin bending channel.

13. A multiple clip package as defined in claim 12, further comprising a guide mechanism for pushing said shaft head inserted in said connection opening, for fastening to said fastening mechanism.

14. A multiple clip package as defined in claim 13, wherein said guide mechanism is a slider for sliding transversely to said axial direction of said multiple clip assembly in said connection opening positioned between said stage portion and said fin bending channel, and for pushing said shaft head in said connection opening upon sliding, to fasten said shaft head with said fastening mechanism.

15. A multiple clip package as defined in claim 14, wherein said slider includes a receiving bore having a diameter substantially equal to a diameter of said fin bending channel.

16. A multiple clip package as defined in claim 15, wherein said receiving bore is disposed between said fin bending channel and said stage portion when said slider is slid for fastening said shaft head to said fastening mechanism.

17. A multiple clip package as defined in claim 16, wherein said housing is cylindrical and has an outer diameter substantially equal to an outer diameter of said flexible sheath, and said stage portion is loaded with said flexible sheath after removal of said housing.

18. A multiple clip package as defined in claim 17, wherein an opening of a sheath end of said flexible sheath on said stage portion is positioned in registration with said fin bending channel, and said multiple clip assembly pulled with said operating wire is loaded in said flexible sheath by advancing a proximal end thereof in a state of stowing said fin portion.

19. A multiple clip package as defined in claim 18, wherein said coupling device includes:
a stage groove formed for causing said housing or said flexible sheath on said stage portion to appear externally at least partially;
a recess, formed in a peripheral portion of said stage groove, for protruding a portion of said housing or said flexible sheath appearing at least partially.

20. A multiple clip package as defined in claim 6, wherein said tubular shells are disposed around respectively said clips, partially cover a rear one of said clips for maintaining a fastened state thereof, and are prevented from moving backwards by said rear clip.

21. A multiple clip package as defined in claim 20, wherein said fin portion pushes and retains one of said clips in said tubular shells when depressed and stowed.

22. A multiple clip package as defined in claim 6, further comprising:
an engaging portion disposed with a first one of said housing and said stage portion;
a receiving portion, disposed with a second one of said housing and said stage portion, for preventing said housing from rotating with respect to said coupling device by engagement with said engaging portion.

23. A multiple clip package as defined in claim 22, wherein a first one of said engaging portion and said receiving portion is a key projection disposed on said housing to project, and a second one of said engaging portion and said receiving portion is a key way groove formed in said stage portion to extend in an axial direction of said housing.

24. A multiple clip package as defined in claim 23, further comprising a retaining mechanism for positioning said key projection engaged with said key way groove in a predetermined position in said axial direction, for retaining said housing to prevent separation in said axial direction.

25. A multiple clip package as defined in claim 24, wherein said retaining mechanism includes:
a retaining projection formed to project from a first one of said key projection and said key way groove;
a retaining hole or recess formed with a second one of said key projection and said key way groove.

26. A multiple clip package as defined in claim 8, wherein said shank has a shape of a section engageable with said pull opening rotationally with respect to an axial direction of insertion thereof.

27. A multiple clip package as defined in claim 26, wherein said shank has resiliency for absorbing twisting of said pull tab portion with respect to said axial direction between said pull tab portion and said pull opening.

28. A multiple clip package as defined in claim 27, wherein said shank includes at least two resilient elongated plates, disposed to extend in said axial direction, and arranged at a predetermined interval in a direction transverse to said axial direction, and a shape of a section of said shank defined by a contour of said elongated plates is similar to a shape of said pull opening.

29. A multiple clip package as defined in claim 28, wherein said elongated plates have such a form that said shank is engaged with said pull opening rotationally when said elongated plates are deformed toward one another.

30. A multiple clip package as defined in claim 8, further comprising:
a retainer disposed in said pull opening; and
a receiving portion, disposed with said shank, for retaining said clip moving structure by engagement with said retainer when said clip moving structure is in a predetermined position with respect to said coupling device.

31. A multiple clip package as defined in claim 30, wherein said coupling device includes a releasing portion for disengaging said first clip from said end connector when a fastening mechanism at a proximal end of said multiple clip assembly reaches a position for enabling fastening to an operating wire in said flexible sheath;
said receiving portion is positioned for engaging with said retainer in operation of said releasing portion.

32. A multiple clip package as defined in claim 30, wherein said receiving portion is positioned for engaging with said retainer when said clip moving structure is pulled to an end position in said coupling device.

33. A multiple clip package as defined in claim 30, wherein said receiving portion is positioned for engaging with said retainer when said clip moving structure is in an initial position before pull relative to said coupling device.

34. A multiple clip package as defined in claim 30, wherein said shank includes at least two resilient elongated plates, disposed to extend in an axial direction of insertion thereof, and arranged at a predetermined interval in a direction transverse to said axial direction;
said receiving portion includes a retaining hole formed in at least one of said elongated plates, and said retainer includes a projection for engaging with said retaining hole, and for disengagement from said retaining hole when said elongated plates resiliently deform in a direction toward one another.
